(19) United States
(12) Patent Application Publication
(10) Pub. No.: US 2023/0321235 A1
(43) Pub. Date: Oct. 12, 2023

(54) VETO CAR-T CELLS

(71) Applicants: Yeda Research and Development Co. Ltd., Rehovot (IL); Ichilov Tech Ltd., Tel-Aviv (IL)

(72) Inventors: Yair REISNER, Houston, TX (US); Anat GLOBERSON LEVIN, Tel-Aviv (IL); Esther BACHAR-LUSTIG, Rehovot (IL); Assaf LASK, Rehovot (IL); Bar NATHANSOHN-LEVI, Rehovot (IL); Tova WAKS, Rehovot (IL); Galit HORN, Hod Hasharon (IL); Zelig ESHHAR, Rehovot (IL)

(73) Assignees: Yeda Research and Development Co. Ltd., Rehovot (IL); Ichilov Tech Ltd., Tel-Aviv (IL)

(21) Appl. No.: 18/020,441

(22) PCT Filed: Aug. 11, 2021
(86) PCT No.: PCT/IL2021/050984

§ 371 Date: Feb. 9, 2023

Related U.S. Application Data
(60) Provisional application No. 63/064,038, filed on Aug. 11, 2020.

(51) Int. Cl. A61P 35/00; C12N 5/0783; A61K 39/00

(52) U.S. Cl. CPC A61K 39/4611; A61K 39/4631; A61P 35/00; C12N 5/0636; A61K 2239/22; C12N 2510/00

(57) ABSTRACT

A method of generating a population of genetically modified veto cells is disclosed. The method comprising: (a) providing a population of cells comprising T cells, the T cells comprising at least 40% memory CD8⁺ T cells; (b) culturing the population of cells comprising T cells with an antigen or antigens under conditions which allow enrichment of tolerance-inducing antigen-specific cells having a central memory T-lymphocyte (Tcm) phenotype, the cells being depleted of graft versus host (GVH) reactivity; and (c) transducing the cells with a polynucleotide encoding a heterologous cell surface receptor comprising a T cell receptor signaling module, thereby generating the population of genetically modified veto cells.

### RELATED APPLICATIONS

This application claims the benefit of priority of U.S. Provisional Pat. Application No. 63/064,038 filed on Aug. 11, 2020, the contents of which are incorporated herein by reference in their entirety.

### SEQUENCE LISTING STATEMENT

The ASCII file, entitled 88373 Sequence Listing.txt, created on 10 Aug. 2021, comprising 8,192 bytes, submitted concurrently with the filing of this application is incorporated herein by reference.

### FIELD AND BACKGROUND OF THE INVENTION

The present invention, in some embodiments thereof, relates to genetically modified veto cells generated from memory T cells and transduced to express a cell surface receptor and, more particularly, but not exclusively, to methods of their manufacture and to the use of same in immunotherapy.

Adoptive cell therapy (ACT) is a therapeutic procedure in which lymphocytes (e.g. T cells) are administered to patients in order to treat cancer or viral infections. A major objective is to apply ACT, including genetically modified T cells, using fully or partially mismatched allogeneic cells without resorting to immature hematopoietic cell transplantation. Alternatively, ACT can allow transplantation of immature hematopoietic cells without prior eradication of the subject’s complete immune system. Thus, ACT can help overcome the primary transplant-related cause of mortality associated with the critical immune reconstitution period (i.e. the dangers faced by a patient with poor immune protection during the early post-transplant period, including the risk of graft versus host disease (GvHD)).

This approach requires the ex vivo generation of tumor- or viral-specific T cells and infusion of same to patients. In order to support the acceptance of the T cells, the patient is typically also treated with conditioning protocols, for example, preconditioning protocols (e.g. irradiation or chemotherapy) and/or administration of lymphocyte growth factors (such as IL-2). Many methods have been described for generating tumor specific lymphocytes with the two main approaches being expansion of antigen specific T cells or redirection of T cells using genetic engineering.

Gene modification approach is used to redirect lymphocytes against tumors via the use of transgenic TCR chains or chimeric receptors. Currently, retroviral or lentiviral, or electroporational transfer of chimeric antigen receptors (CARs) whose target recognition is dependent on a single-chain variable region domain of a monoclonal antibody (scFv) or that of a T-cell receptor (TCR) is typically used for stable production of therapeutic T cells (CAR-T cells or TCR-T cells, respectively) [Fujiwara, Pharmaceuticals (2014) 7: 1049-1068].

CAR-T cells are not HLA restricted. The construct of the chimeric receptor (chimeric antigen receptor - CAR) is typically composed of an extracellular antigen-binding domain, a transmembrane domain and a cytoplasmic signaling domain. The original chimeric receptor (i.e. ‘first-generation’) was composed of a scFv fragment fused to an intracellular domain from the CD3 ζ- chain. A ‘second generation’ chimeric receptor was also generated which adds an intracellular signaling domain, from various co-stimulatory protein receptors (e.g. CD28, CD137, 4-1BB, ICOS), to the cytoplasmic tail of the CAR to provide additional signals to the T cell. Preclinical studies have indicated that the ‘second generation’ CARs improved the anti-tumor activity of T cells. A “third-generation” CARs was recently generated which combine multiple signaling domains, such as CD3zeta-CD28-4-1BB or CD3zeta-CD28-OX40, to further augment potency.

Tumor specific CARs targeting a variety of tumor antigens are being tested in the clinic for treatment of a variety of different cancers. Examples of these cancers and their antigens that are being targeted includes follicular lymphoma (CD20 or GD2), neuroblastoma (CD171), non-Hodgkin lymphoma (CD20), lymphoma (CD19), glioblastoma (IL13Rα2), chronic lymphocytic leukemia or CLL and acute lymphocytic leukemia or ALL (both CD19). CARs demonstrating activity against solid tumors including ovarian, prostate, breast, renal, colon, neuroblastoma and others are under investigation. Virus specific CARs have also been developed to attack cells harboring virus such as HIV. For example, a clinical trial was initiated using a CAR specific for Gp100 for treatment of HIV (Chicaybam, Ibid).

Various approaches have been contemplated for modifying T-cells for adoptive cell therapy, some are described in Gilham et al., Human Gene Therapy (2015) 26:276-285; in Sharpe and Mount, Disease Models and Mechanisms (2015) 8:337-350 and in Gouble et al. Blood (2014) 124(21) 4689.

Various approaches have been contemplated for generation of genetically modified tolerance inducing T-lymphocytes transduced to express a cell surface receptor and devoid of graft versus host (GVH) activity and the use of same for immunotherapy, some are summarized infra.

PCT publication no. WO 2018/134824 discloses an isolated cytotoxic T-lymphocyte (CTL), the CTL being tolerance inducing and substantially depleted of alloreactivity, and wherein the CTL is transduced to express a cell surface receptor comprising a T cell receptor signaling module (e.g. chimeric antigen receptor, i.e. CAR). Methods of generating these CTLs and use of same in therapy are also disclosed.

PCT publication no. WO 2017/009853 discloses an isolated cell having a central memory T-lymphocyte (Tcm) phenotype, the Tcm being tolerance-inducing and capable of homing to the lymph nodes following transplantation, and wherein the Tcm is transduced to express a cell surface receptor comprising a T cell receptor signaling module (e.g. chimeric antigen receptor, i.e. CAR). Methods of generating these Tcm cells and use of same in therapy are also disclosed.

PCT publication no. WO/2018/002924 discloses an isolated population of non-GvHD inducing cells comprising a Tcm phenotype, the cells being tolerance inducing cells and/or endowed with anti-disease activity, and capable of homing to the lymph nodes following transplantation. The Tcm cells taught by WO/2018/002924 are generated from memory T cells by depleting alloreactive clones from memory T cells by way of antigen activation. Methods of generating these Tcm cells and use of same in therapy are also disclosed.

### SUMMARY OF THE INVENTION

According to an aspect of some embodiments of the present invention there is provided a method of generating a population of genetically modified veto cells, the method comprising: (a) providing a population of cells comprising T cells, the T cells comprising at least 40% memory CD8⁺ T cells; (b) culturing the population of cells comprising T cells with an antigen or antigens under conditions which allow enrichment of tolerance-inducing antigen-specific cells having a central memory T-lymphocyte (Tcm) phenotype, the cells being depleted of graft versus host (GVH) reactivity; and (c) transducing the cells with a polynucleotide encoding a heterologous cell surface receptor comprising a T cell receptor signaling module, thereby generating the population of genetically modified veto cells.

According to some embodiments of the invention, step (b) is affected concomitantly with step (c).

According to some embodiments of the invention, step (b) is affected prior to step (c).

According to some embodiments of the invention, step (c) is affected prior to step (b).

According to an aspect of some embodiments of the present invention there is provided an isolated population of genetically modified veto cells obtainable according to the method of some embodiments of the invention.

According to an aspect of some embodiments of the present invention there is provided a pharmaceutical composition comprising the isolated population of genetically modified veto cells of some embodiments of the invention and a pharmaceutically active carrier.

According to an aspect of some embodiments of the present invention there is provided a method of treating a disease in a subject in need thereof, the method comprising administering to the subject a therapeutically effective amount of the isolated population of genetically modified veto cells of some embodiments of the invention, thereby treating the subject.

According to an aspect of some embodiments of the present invention there is provided a therapeutically effective amount of the isolated population of genetically modified veto cells of some embodiments of the invention for use in treating a disease in a subject in need thereof.

According to an aspect of some embodiments of the present invention there is provided a method of treating a disease in a subject in need thereof, the method comprising: (a) analyzing a biological sample of a subject for the presence of an antigen or antigens associated with the disease; (b) generating genetically modified veto cells according to the method of some embodiments of the invention towards the antigen or antigens associated with the disease; and (c) administering to the subject a therapeutically effective amount of the genetically modified veto cells of step (b), thereby treating the disease in the subject.

According to an aspect of some embodiments of the present invention there is provided a method of treating a subject in need of a cell or tissue transplantation, the method comprising: (a) transplanting a cell or tissue transplant into the subject; and (b) administering to the subject an effective amount of the isolated population of genetically modified veto cells of some embodiments of the invention, thereby treating the subject in need of the cell or tissue transplantation.

According to an aspect of some embodiments of the present invention there is provided a therapeutically effective amount of the isolated population of genetically modified veto cells of some embodiments of the invention for use as an adjuvant treatment for a cell or tissue transplant into a subject, wherein the subject is in need of a cell or tissue transplantation.

According to some embodiments of the invention, step (a) is affected by treating peripheral blood mononuclear cells (PBMCs): (i) with an agent capable of depleting CD4⁺, CD56⁺ and CD45RA⁺ cells; or (ii) with an agent capable of selecting CD45RO⁺, CD8⁺ cells, so as to obtain a population of cells comprising T cells enriched of memory CD8⁺ T cells comprising a CD45RO⁺CD45RA⁻CD8⁺ phenotype.

According to some embodiments of the invention, the heterologous cell surface receptor comprises a chimeric antigen receptor (CAR) or a transgenic T cell receptor (tg-TCR).

According to some embodiments of the invention, the CAR comprises at least one co-stimulatory domain.

According to some embodiments of the invention, the at least one co-stimulatory domain is selected from the group consisting of CD28, CD134/OX40, CD137/4-1BB, Lck, ICOS and DAP10.

According to some embodiments of the invention, the CAR comprises at least one signaling domain.

According to some embodiments of the invention, the signaling domain comprises a CD3ζ or a FcR-γ.

According to some embodiments of the invention, the CAR comprises at least one of a transmembrane domain and a hinge domain.

According to some embodiments of the invention, the transmembrane domain is selected from a CD8 and a CD28.

According to some embodiments of the invention, the hinge domain is selected from a CD8 and a CD28.

According to some embodiments of the invention, the CAR comprises an antigen binding domain being an antibody or an antigen-binding fragment.

According to some embodiments of the invention, the antigen-binding fragment is a Fab or a scFv.

According to some embodiments of the invention, the CAR or the tg-TCR binds an antigen selected from the group consisting of a tumor antigen, a viral antigen, a bacterial antigen, a fungal antigen, a protozoa antigen, and a parasite antigen.

According to some embodiments of the invention, the tumor antigen is associated with a solid tumor.

According to some embodiments of the invention, the tumor antigen is associated with a hematologic malignancy.

According to some embodiments of the invention, the antigen or antigens targeted by the veto cell is distinct from the antigen targeted by the CAR or by the tg-TCR.

According to some embodiments of the invention, the memory CD8⁺ T cells are devoid of CD45RA⁺ cells.

According to some embodiments of the invention, the memory CD8⁺ T cells are devoid of CD4⁺ and/or CD56⁺ cells.

According to some embodiments of the invention, the memory CD8⁺ T cells comprise a CD45RO⁺CD45RA⁻CD8⁺ phenotype.

According to some embodiments of the invention, the culturing the population of cells comprising T cells with an antigen or antigens under conditions which allow enrichment of tolerance-inducing antigen-specific cells having a Tcm phenotype, is affected by a method comprising: (a) contacting the population of cells comprising T cells with the antigen or antigens in the presence of IL-21 so as to allow enrichment of antigen reactive cells; and (b) culturing the cells resulting from step (a) in the presence of IL-21, IL-15 and/or IL-7 so as to allow proliferation of cells comprising the Tcm phenotype.

According to some embodiments of the invention, the antigen or antigens comprise third-party antigen or antigens.

According to some embodiments of the invention, the antigen or antigens is selected from the group consisting of a viral antigen, a bacterial antigen, a tumor antigen, a protein extract, a purified protein and a synthetic peptide.

According to some embodiments of the invention, the viral antigen comprises one or more viral peptides.

According to some embodiments of the invention, the viral antigen comprises an EBV peptide, a CMV peptide, an Adenovirus (Adv) peptide and/or a BK virus peptide.

According to some embodiments of the invention, the antigen or antigens is presented by antigen presenting cells, artificial vehicles or artificial antigen presenting cells.

According to some embodiments of the invention, the antigen or antigens is presented by antigen presenting cells of the same origin as the population of cells comprising T cells.

According to some embodiments of the invention, the antigen presenting cells are dendritic cells.

According to some embodiments of the invention, contacting the population of cells comprising T cells with the antigen or antigens in the presence of IL-21 is affected for 12 hours to 6 days.

According to some embodiments of the invention, contacting the population of cells comprising T cells with the antigen or antigens in the presence of IL-21 is affected for 3 days.

According to some embodiments of the invention, culturing the cells resulting from step (a) in the presence of IL-21, IL-15 and/or IL-7 is affected for 12 hours to 20 days.

According to some embodiments of the invention, culturing the cells resulting from step (a) in the presence of IL-21, IL-15 and IL-7 is affected for 4 days to 12 days.

According to some embodiments of the invention, culturing the cells resulting from step (a) in the presence of IL-21, IL-15 and IL-7 is affected for 9 days.

According to some embodiments of the invention, the total length of time for generating the tolerance-inducing antigen-specific cells having a Tcm phenotype is 12 days of culture.

According to some embodiments of the invention, the method further comprises depleting alloreactive cells following step (b).

According to some embodiments of the invention, depleting alloreactive cells is affected by depletion of CD137⁺ and/or CD25⁺ cells following contacting the cells comprising the Tcm phenotype with host antigen presenting cells (APCs).

According to some embodiments of the invention, the method is affected ex-vivo.

According to some embodiments of the invention, the Tcm phenotype comprises a CD3⁺, CD8⁺, CD62L⁺, CD45RA⁻, CD45RO⁺ signature.

According to some embodiments of the invention, at least 50% of the cells are CD3⁺CD8⁺ cells of which at least 30% have the signature.

According to some embodiments of the invention, transducing is affected on days 3-7 of culture.

According to some embodiments of the invention, transducing is with a polynucleotide encoding the CAR or the tg-TCR.

According to some embodiments of the invention, the genetically modified veto cells are endowed with anti-viral activity.

According to some embodiments of the invention, the genetically modified veto cells are endowed with anti-disease activity.

According to some embodiments of the invention, the genetically modified veto cells are endowed with anti-tumor activity.

According to some embodiments of the invention, the population of genetically modified veto cells comprises a diverse population of cells.

According to some embodiments of the invention, at least 10% of the veto cells within the population of cells express the heterologous cell surface receptor.

According to some embodiments of the invention, the method further comprises transplanting a cell or tissue transplant into the subject.

According to some embodiments of the invention, the therapeutically effective amount of the isolated population of genetically modified veto cells further comprises a cell or tissue transplant.

According to some embodiments of the invention, the transplanting is affected concomitantly with, prior to, or following administering of the genetically modified veto cells.

According to some embodiments of the invention, the genetically modified veto cells are for administration prior to, concomitantly with, or following the cell or tissue transplant.

According to some embodiments of the invention, the disease is selected from the group consisting of a malignant disease, a viral disease, a bacterial disease, a fungal disease, a protozoa disease, and a parasite disease.

According to some embodiments of the invention, the malignant disease is a solid tumor or tumor metastasis.

According to some embodiments of the invention, the malignant disease is a hematological malignancy.

According to some embodiments of the invention, the hematological malignancy comprises a leukemia, a lymphoma or multiple myeloma.

According to some embodiments of the invention, the malignant disease is selected from the group consisting of a leukemia, a lymphoma, a myeloma, a melanoma, a sarcoma, a neuroblastoma, a colon cancer, a colorectal cancer, a breast cancer, an ovarian cancer, an esophageal cancer, a synovial cell cancer and a pancreatic cancer.

According to some embodiments of the invention, the viral disease is caused by a virus selected from the group consisting of an Epstein-Barr virus (EBV), a Cytomegalovirus (CMV), an Adenovirus (Adv), a BK virus, an immunodeficiency virus (HIV), an influenza, a T-cell leukemia virus type 1 (TAX), a hepatitis C virus (HCV), a hepatitis B virus (HBV) and severe acute respiratory syndrome coronavirus 2 (SARS-CoV-2).

According to some embodiments of the invention, the genetically modified veto cells are non-syngeneic with the subject.

According to some embodiments of the invention, the method further comprises conditioning the subject under sublethal, lethal or supralethal conditioning protocol prior to administering.

According to some embodiments of the invention, the therapeutically effective amount for use further comprises a sublethal, lethal or supralethal conditioning protocol.

According to some embodiments of the invention, the sublethal, lethal or supralethal conditioning is selected from the group consisting of a myeloablative conditioning, a non-myeloablative conditioning, a co-stimulatory blockade, a chemotherapeutic agent, an irradiation therapy and an immunotherapy.

According to some embodiments of the invention, the non-myeloablative conditioning comprises T cell debulking.

According to some embodiments of the invention, the T cell debulking is affected by antibodies selected from the group consisting of anti-thymocyte globulin (ATG) antibodies, anti-CD52 antibodies and anti-CD3 (OKT3) antibodies.

According to some embodiments of the invention, the administering is effected by a route selected from the group consisting of intratracheal, intrabronchial, intraalveolar, intravenous, intraperitoneal, intranasal, subcutaneous, intramedullary, intrathecal, intraventricular, intracardiac, intramuscular, intraserosal, intramucosal, transmucosal, transnasal, rectal and intestinal.

According to some embodiments of the invention, the subject is a human subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a schematic illustration of a protocol of some embodiments of the invention for generating T central memory veto cells.

FIG. 1B is illustrates the CAR retroviral vector, i.e. scFv N29 CAR cloned into the pBullet retroviral vector.

FIG. 2 illustrates a marked depletion of alloreactivity in anti-viral veto Tcm cells generated from memory T cells co-cultured with dendritic cells (DCs) loaded with viral peptides for third-party stimulation. Veto Tcm cells were prepared from CD4⁻CD56⁻ (presented in blue/diamonds, i.e. a combined population of naïve and memory T cells) or from CD4⁻CD56⁻CD45RA⁻ (presented in green/triangles, i.e. memory T cells) donor responder cells which were co-cultured on day 3 against irradiated autologous DCs pulsed with viral peptides derived from EBV, CMV, BKV and Adenovirus in the presence of IL-21 for 3 days, with the addition of IL-21, IL-15 and IL-7 from days 3-15. On day 15, a control population of fresh CD4⁻CD56⁻CD19⁻ cells (presented in red/squares) was bead-sorted from freshly thawed donor cells. All three cell preparations (i.e. anti-viral veto Tcm CD4⁻CD56⁻, anti-viral veto Tcm CD4⁻CD56⁻CD45RA⁻ and fresh CD4⁻CD56⁻CD19⁻ cells) were cultured against irradiated host peripheral blood mononuclear cells (PBMCs) for 5 days and then harvested and re-stimulated for 6 days against irradiated host PBMCs in limiting dilution assay (LDA) in the presence of IL-2 for the induction of an effector phenotype. On day 21, S³⁵-Methionine LDA killing assay was carried out against concanavalin A (ConA) blasts host origin. After 5 hours, Mixed Lymphocyte Reaction (MLR), supernatant was collected from wells and sentilation liquid was added. A linear regression plot of % non-responding cultures versus cell number per culture is presented. The frequency (f) of anti- host clones in the specific culture was calculated from the linear regression slope.

FIGS. 3A-E illustrate the veto activity of veto CAR-T cells. (FIG. 3A) Phenotype of transduced veto cells; (FIG. 3B) Expression of N29 CAR-GFP in transduced veto cells as measured by FACS at day 12 of culture; (FIG. 3C) Veto CAR-T cells exhibit similar reactivity to that exhibited by regular transduced T cells, against Her2 positive target cells (SKOV- ovarian cancer cell line), as measured by secretion of IFN-γ; (FIG. 3D) After CAR transduction, veto CAR-T cells exhibit marked veto activity measured following addition of donor derived veto cells to mixed lymphocyte reaction (MLR) of responder cells against donor type stimulator (specific veto at a ratio of 1:5) compared to addition of donor derived veto cells to MLR of responder cells against 3^{rd} party stimulators (non-specific veto at 1:5 ratio). Frequency of alloreactive clones at the end of culture is defined by measuring the ability to detect responding cells at different responder concentrations following limit dilution analysis. For each frequency determination, the line represents the estimate Minimum x²; (FIG. 3E) The estimated number of cells required for detection in culture of one alloreactive clone (based on limit dilution analysis shown in FIG. 3D). Non-specific culture represents addition of veto cells to MLR against 3^{rd} party stimulator. Specific culture represents addition of donor derived veto cells to MLR against the donor.

FIGS. 4A-C illustrate the anti-viral activity of veto Tcm cells as determined by intracellular staining of INF-γ and TNF-α. Veto-Tcm cells were co-cultured with the respective viral peptide mix (EBV, CMV, Adeno, BKV) in the presence of Brefeldin A (eBioscience) at 37° C., 5% CO₂ for 6 hrs. Cells were fixed, permeabilized (Invitrogen Fix & Perm set), and immunostained for CD45, CD3, CD8, IFN-γ, and TNF-α (BD). Positive control included TCR-independent stimulation with PMA/Ionomycin. Cells were gated on a CD45+/FSC lymphocyte gate and CD3⁺CD8⁺.

FIG. 5 illustrates calibration of the VETO-CAR cells transduction. VETO cells were transduced with a vector at the indicated days to express a chimeric antigen receptor (i.e. anti-ErbB-2 (N29)) also expressing the reporter GFP. Transduction percentage of veto-CAR cells was analyzed using CD3 staining for T cells and GFP expression as CAR T indication.

### DESCRIPTION OF SPECIFIC EMBODIMENTS OF THE INVENTION

The present invention, in some embodiments thereof, relates to genetically modified veto cells generated from memory CD8⁺ T cells and transduced to express a cell surface receptor and, more particularly, but not exclusively, to methods of their manufacture and to the use of same in immunotherapy.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The goal of allowing transplantation of a new immune system without complete eradication of the old one is very desirable for cancer patients. This addresses the primary transplant related causes of mortality associated with the critical immune reconstitution period (i.e. the dangers faced by a patient with poor immune protection during the early post-transplant period), including the risk of graft versus host disease (GvHD).

Cell-based therapies with lymphocytes and antigen-presenting cells are promising approaches for immunotherapy (including cancer therapy). Adoptive cell transfer (ACT), including transfer of immune-derived cells such as CAR-T cells, offers the goal of transferring the immunologic functionality and characteristics into the host. However, the problem of graft rejection (by the transplant recipient) and/or GvHD (by the transplanted cells) is an ongoing problem that needs to be overcome in order to pursue therapeutic potential of these cells.

While reducing the present invention to practice, the present inventors have uncovered that veto central memory T (Tcm) cells generated from the T cell memory pool of a donor and cultured against viral antigens under cytokine deprivation during the first 3 days of culture, are endowed with intrinsic veto tolerance inducing activity (e.g. avoiding graft rejection), comprise anti-viral activity, and do not induce graft versus host disease (GvHD). The present inventors further discovered that the veto Tcm cells can be genetically modified to express a T cell receptor (e.g. transgenic T cell receptor or a chimeric antigen receptor) and can be used to combat disease (e.g. tumor) while maintaining their veto cell properties.

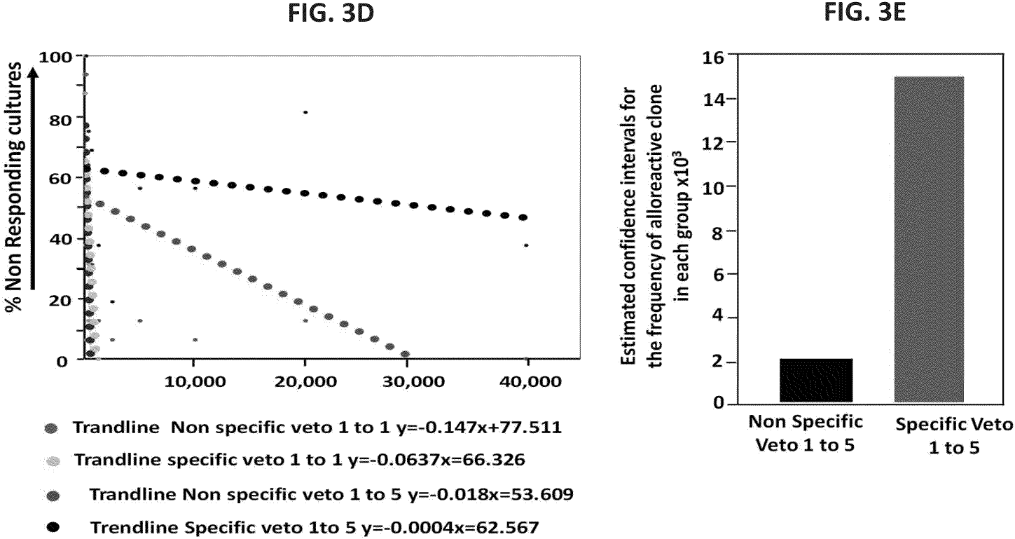

As is shown hereinbelow and in the Examples section which follows, the present inventors have shown that veto CAR-T cells can be produced within a few days (e.g. 9 days) by first generating central memory CD8⁺ veto cells from the T cell memory pool of a donor using stimulation against donor dendritic cells (DCs) pulsed with viral peptides (EBV, CMV, Adenovirus and BK virus, see Example 1 hereinbelow), and then transducing the cells with a retroviral supernatent (e.g. on day 5 of culture) to express a chimeric antigen receptor (e.g. scFv N29 CAR, see Example 2, hereinbelow). This protocol yields high levels of CAR expression in human anti-viral CD8⁺ veto cells. Specifically, the veto cell product harvested at the end of culture (e.g. on day 12 of culture) exhibited more than 90% CD8⁺ T cells (see FIG. 3A) of which more than 70% expressed the transfected CAR (e.g. anti-Her2 CAR, see FIG. 3B). Importantly, the veto CAR-T cells exhibited their veto effect (see FIGS. 3D-E) and exhibited specific reactivity against their target antigen (e.g. Her2 antigen, see FIG. 3C).

The veto CAR-T cells of the invention can be used in the context of hematopoietic stem cell transplantation (HSCT), specifically in the context of non-myeloablative haploidentical megadose T cell depleted transplantation, enabling to overcome graft rejection while preventing severe graft versus host disease and actively killing cancer cells. Notably, non-myeloablative conditioning does not ablate all of the recipient subject’s immune system (including T cells) and thereby offers immune protection against pathogens until the newly formed donor-derived T cells emerge from the thymus. Moreover, as the veto CAR-T cells of the invention are expanded in culture against viral antigens, they further provide protection from viral infection, e.g. Epstein-Barr virus (EBV), cytomegalovirus (CMV), Adenovirus and BK virus, known to have adverse impact post-HSCT. The veto CAR-T cells of the invention can be further used without HSCT for the treatment of patients with hematological malignancies such as those in relapse.

Taken together, the veto CAR-T cells of the invention offer the solution of (1) being tolerance inducing cells (avoiding graft rejection and GvHD), (2) comprising anti-viral activity, and (3) anti-tumor activity, all in a single cell. Accordingly, the veto CAR-T cells can be used alone for disease treatment, or can be used along with a hematopoietic stem cell transplant (e.g. haploidentical HSCT) to facilitate engraftment and disease treatment. Moreover, these cells overcome the need of manufacturing the cell based therapies on a “per patient basis” and enable manufacture of an “off-the-shelf” product for therapy.

Thus, according to one aspect of the present invention there is provided a method of generating a population of genetically modified veto cells, the method comprising: (a) providing a population of cells comprising T cells, the T cells comprising at least 40% memory CD8⁺ T cells; (b) culturing the population of cells comprising T cells with an antigen or antigens under conditions which allow enrichment of tolerance-inducing antigen-specific cells having a central memory T-lymphocyte (Tcm) phenotype, the cells being depleted of GVH reactivity; and (c) transducing the cells with a polynucleotide encoding a heterologous cell surface receptor comprising a T cell receptor signaling module, thereby generating the population of genetically modified veto cells.

According to another aspect of the present invention, there is provided a method of generating a population of genetically modified veto cells, the method comprising: (a) treating peripheral blood mononuclear cells (PBMCs) with an agent capable of depleting CD4⁺, CD56⁺ and CD45RA⁺ cells, or with an agent capable of selecting CD45RO⁺, CD8⁺ cells, so as to obtain a population of cells comprising T cells enriched of memory CD8⁺ T cells comprising a CD45RO⁺CD45RA⁻CD8⁺ phenotype; (b) culturing the population of cells comprising T cells with an antigen or antigens under conditions which allow enrichment of tolerance-inducing antigen-specific cells having a central memory T-lymphocyte (Tcm) phenotype the cells being depleted of GVH reactivity; and (c) transducing the cells with a polynucleotide encoding a heterologous cell surface receptor comprising a T cell receptor signaling module, thereby generating the population of genetically modified veto cells.

The phrase “tolerance inducing cells” as used herein refers to cells which provoke decreased responsiveness of the recipient’s cells (e.g. recipient’s T cells) when they come in contact with the donor cells as compared to the responsiveness of the recipient’s cells in the absence of administered tolerance inducing cells. Tolerance inducing cells include veto cells (i.e. T cells which lead to apoptosis of host T cells upon contact with same) as was previously described in PCT Publication Nos. WO 2001/049243 and WO 2002/102971.

The term “veto activity” relates to immune cells (e.g. donor derived T cells) which lead to inactivation of anti-donor recipient T cells upon recognition and binding to the veto cells. According to one embodiment, the inactivation results in apoptosis of the anti-donor recipient T cells. It will be appreciated that veto cells typically exert their tolerance-inducing activity based on the T cell receptor of the responding T cells, i.e. T cells directed against the veto cells. Thus, veto activity results from unidirectional recognition of the veto cell by the responding T cell, but not vice versa. In addition, the veto cells typically exert their activity in a T cell receptor (TCR)-independent mechanism (as previously discussed in Ophir and Reisner, Front Immunol. (2012) 93(3): 1-6; and in Arditti et al., Blood (2005) 105(8):3365-71. Epub 2004 Jul 6).

The tolerance-inducing cells of some embodiments of the invention are also referred to herein as “veto cells”.

The veto cells of some embodiments of the invention comprise a Tcm phenotype.

The phrase “central memory T-lymphocyte (Tcm) phenotype” as used herein refers to a subset of T cells which home to the lymph nodes. Cells having the Tcm phenotype, in humans, typically comprise a CD3⁺/CD8⁺/CD62L⁺/CD45RO⁺/CD45RA⁻ signature. It will be appreciated that Tcm cells may express all of the signature markers on a single cell or may express only part of the signature markers on a single cell. Determination of a cell phenotype can be carried out using any method known to one of skill in the art, such as for example, by Fluorescence-activated cell sorting (FACS) or capture ELISA labeling.

According to one embodiment, at least 20%, at least 25%, at least 30%, at least 35%, at least 40%, at least 50%, at least 55%, at least 60%, at least 65%, at least 70%, at least 75%, at least 80%, at least 85%, at least 90%, at least 95%, at least 99%, or even 100% of the genetically modified veto cells have the Tcm cell signature.

According to a specific embodiment, about 10-20%, about 10-30%, about 10-40%, about 10-50%, about 20-30%, about 20-40%, about 30-40%, about 30-50%, about 40-50%, about 40-60%, about 50-60%, about 50-70%, about 60-70%, about 60-80%, about 70-80%, about 70-90%, about 80-90%, about 80-100%, or about 90-100% of the genetically modified veto cells have the Tcm cell signature.

According to a specific embodiment, cells having the Tcm phenotype comprise about 10% of the genetically modified veto cells.

According to a specific embodiment, cells having the Tcm phenotype comprise about 30% of the genetically modified veto cells.

According to a specific embodiment, cells having the Tcm phenotype comprise about 50% of the genetically modified veto cells.

According to a specific embodiment, cells having the Tcm phenotype comprise about 70% of the genetically modified veto cells.

According to a specific embodiment, cells having the Tcm phenotype comprise about 80% of the genetically modified veto cells.

According to a specific embodiment, cells having the Tcm phenotype comprise about 90% of the genetically modified veto cells.

The veto cells comprising a Tcm phenotype of the invention are also referred to herein as “Tcm cells” or “veto Tcm cells”.

As mentioned, Tcm cells typically home to the lymph nodes following transplantation. According to some embodiments, cells of some embodiments of the invention may home to any of the lymph nodes following transplantation, as for example, the peripheral lymph nodes and mesenteric lymph nodes. The homing nature of these cells allows them to exert their veto effect in a rapid and efficient manner.

The genetically modified veto cells of some embodiments of the invention are isolated cells.

The term “isolated” refers to at least partially separated from the natural environment e.g., from a cell, or from a tissue, e.g., from a human body.

According to one embodiment, the population of genetically modified cells comprises a homogeneous cell mixture (also referred to as a clonal population).

According to one embodiment, the population of genetically modified cells comprises a heterogeneous cell mixture (also referred to as a diverse population of cells).

According to some embodiments, the genetically modified veto cells are depleted of graft versus host (GVH) reactivity.

According to some embodiments, the genetically modified veto cells are non-GvHD inducing cells.

The term “non-graft versus host” or “non-GVH” as used herein refers to having substantially reduced or no graft versus host disease (GVHD) inducing reactivity. Thus, the cells of some embodiments of the present invention are generated as to not significantly cause GVHD as evidenced by survival, weight and overall appearance of the transplanted subject 30-120 days following transplantation. Methods of evaluating a subject for reduced GVHD are well known to one of skill in the art.

According to one embodiment, the genetically modified veto cells of some embodiments of the invention have at least 10%, at least 20%, at least 30%, at least 40%, at least 50%, at least 55%, at least 60%, at least 65%, at least 70%, at least 75%, at least 80%, at least 85%, at least 90%, at least 95 %, at least 97%, at least 99% or even 100% reduced reactivity against a host relative to cells (e.g. CAR-T cells) not generated according to the present teachings.

Additionally or alternatively, the genetically modified veto cells comprising a Tcm phenotype of some embodiments of the invention comprise anti-disease activity.

The term “anti-disease activity” refers to the function of the veto cells comprising a Tcm phenotype against a diseased cell. The anti-disease activity may be directly against a diseased cell, e.g. killing capability of the diseased cell. This activity may be due to TCR independent killing mediated by LFA1-I/CAM1 binding [Arditti et al., Blood (2005) 105(8):3365-71. Epub 2004 Jul 6]. Additionally or alternatively, the anti-disease activity may be due to genetic modification of the cells (e.g. expression of a T cell receptor such as a chimeric antigen receptor or a transgenic T cell receptor, as discussed below). Additionally or alternatively, the anti-disease activity may be indirect, e.g. by activation of other types of cells (e.g. CD4⁺ T cells, B cells, monocytes, macrophages, NK cells) which leads to death of the diseased cell (e.g. by killing, apoptosis, or by secretion of other factors, e.g. antibodies, cytokines, etc.).

The term “anti-viral activity” refers to the function of the Tcm cells against a virally infected cell (e.g. expressing viral antigen/s in the context of MHC-peptide complex on the cell surface). Typically the anti-viral activity results in killing of the virally infected cell.

The term “anti-tumor activity” refers to the function of the Tcm cells against a tumor cell. Typically the anti-tumor activity results in killing of the tumor cell. According to a specific embodiment, anti-tumor activity comprises graft versus leukemia/lymphoma (GVL) activity.

A diseased cell may comprise, for example, a virally infected cell, a bacterial infected cell, a cancer cell [e.g. cell of a solid tumor or leukemia/lymphoma cell, also referred to herein as graft versus leukemia (GVL) activity of the Tcm cells], or a cell altered due to stress, radiation or age.

According to a specific embodiment, the genetically modified veto cells comprising a Tcm phenotype are endowed with an anti-viral activity (e.g. against a virally infected cell presenting an antigen or antigens recognized by the veto cells by virtue of generation of the Tcm cells, as discussed in detail below).

According to a specific embodiment, the genetically modified veto cells comprising a Tcm phenotype are endowed with an anti-tumor activity (e.g. by virtue of the heterologous cell surface receptor comprising a T cell receptor signaling module, as discussed in detail below).

According to some embodiments, the veto cells of some embodiments of the invention comprising a Tcm phenotype are genetically modified.

The term “genetically modified” refers to cells which are manipulated to express or not express specific genes, markers or peptides or to secrete or not secrete specific cytokines, depending on the application needed (e.g. on the disease to be treated).

According to one embodiment, the veto cells of some embodiments of the present invention comprising a Tcm phenotype are transduced to express a cell surface receptor comprising a T cell receptor signaling module.

As used herein, the term “transduced” may be interchangeably used with the terms “transfected” or “transformed” and refers to a process by which an exogenous nucleic acid (heterologous) is transferred or introduced into a cell. A “transfected” or “transformed” or “transduced” cell is one which has been transfected, transformed or transduced with exogenous nucleic acid. The cell includes the primary cell and its progeny, or cell lines thereof.

The term “cell surface receptor” as used herein refers to a recombinant or synthetic molecule presented on a cell membrane which binds to a ligand (e.g. an antigen) and mediates activation of the cell.

The term “antigen” or “Ag” as used herein is defined as a soluble or non-soluble (such as membrane associated) molecule that provokes an immune response. The skilled artisan will understand that any macromolecule, including virtually all proteins or peptides, as well as carbohydrates, lipids and DNA can serve as an antigen.

According to some embodiments of the invention, the antigen is associated with a malignant disease, i.e. tumor antigen (e.g., tumor specific antigen or a tumor associated antigen), a viral protein antigen, a bacterial protein antigen, or a fungal protein antigen, as described in further detail hereinbelow.

The cell surface receptor of the invention comprises a T cell receptor signaling module.

The term “T cell receptor signaling module” refers to an intracellular portion of the receptor responsible for activation of at least one of the normal effector functions of the T cell in which the receptor has been placed in. Normal effector functions of a T cell may include, for example, secretion of immunostimulatory cytokines (e.g. IFN-gamma, IL-2, TNF-alpha), antigen specific cytotoxicity, and cell proliferation. Thus, the T cell receptor signaling module of the invention refers to the portion of a protein which transduces the effector function signal and directs the cell to perform a specialized function.

According to one embodiment, the cell surface receptor comprises a transgenic T cell receptor (tg-TCR) or a chimeric antigen receptor (CAR).

As used herein, the term “transgenic T cell receptor” or “tg-TCR” refers to a recombinant or synthetic molecule comprising the specificity of a T cell receptor (TCR), i.e. recognition of antigenic peptides (i.e. antigens) presented by major histocompatability complex (MHC) proteins. Typically, the TCR recognizes antigens, i.e. peptides of foreign (e.g. viral) or cellular (e.g. tumor) origins which have been processed by the cell, loaded onto the MHC complex and trafficked to the cell membrane as a peptide-MHC complex.

The tg-TCR of the invention typically comprises two chains (i.e., polypeptide chains), such as, an alpha chain of a T cell receptor (TCR), a beta chain of a TCR, a gamma chain of a TCR, a delta chain of a TCR, or a combination thereof (e.g. αβ chains or γδ chains). The polypeptides of the tg-TCR can comprise any amino acid sequence, provided that the tg-TCR has antigenic specificity and T cell effector functions as described hereinabove. It will be appreciated that antigen specificity is determined by the TCR heterodimer (i.e. by the αβ or γδ chains).

It will be appreciated that each of the two chains is typically composed of two extracellular domains, i.e. the variable (V) region and the constant (C) region.

According to one embodiment, the tg-TCR comprises the variable regions of a TCR. According to a specific embodiment, the tg-TCR comprises the variable regions of α- and β-chains of a TCR. According to another specific embodiment, the tg-TCR comprises the variable regions of γ- and δ-chains of a TCR.

According to some embodiments of the invention, the variable region of the tg-TCR comprises complementarity determining regions (CDRs) which are capable of specifically binding the antigen. The CDRs may be selected from any of CDR1, CDR2, CDR3 and/or CDR4. According to a specific embodiment, the CDRs are present on a single chain, preferably the CDRs are present on both chains of the tg-TCR.

According to one embodiment, the tg-TCR comprises the constant regions of a TCR. According to a specific embodiment, the tg-TCR comprises the constant regions of α- and β-chains of a TCR. According to another specific embodiment, the tg-TCR comprises the constant regions of γ- and δ-chains of a TCR.

In order to avoid formation of mixed dimmers between endogenous TCRs (i.e. TCRs originating within the transduced cell) and the tg-TCR chains, the tg-TCR of the invention may comprise the constant region a murine (e.g. mouse) TCR. Another approach which may be used to increase the specific pairing of tg-TCR chains is to introduce additional cysteine residues within the constant region of the tg-TCR chains (e.g. α and β chains), this results in formation of an additional disulfide bond. Alternatively, mutational inversions of the critical interacting amino acids in the tg-TCR chain (e.g. α and β chain) constant regions may be introduced which favor the pairing of the tg-TCR chains and also increase tg-TCR reactivity. Alternatively or additionally, downregulation of the endogenous TCR may be implemented using, for example, small interfering RNA (siRNA) which is used to specifically down-regulate the endogenous TCR. For further details, see e.g. Zhang and Morgan, Adv Drug Deliv Rev. (2012) 64(8): 756-762, incorporated herein by reference.

As mentioned, the tg-TCR recognizes an antigen in an MHC dependent manner.

As used herein the phrase “major histocompatibility complex” or “MHC” refers to a complex of antigens encoded by a group of linked loci, which are collectively termed H-2 in the mouse and human leukocyte antigen (HLA) in humans. The two principal classes of the MHC antigens, class I and class II, each comprise a set of cell surface glycoproteins which play a role in determining tissue type and transplant compatibility.

The main MHC class I molecules are contemplated herein.

Major histocompatibility complex (MHC) class I molecules are expressed on the surface of nearly all cells. These molecules function in presenting peptides which are mainly derived from endogenously synthesized proteins to CD8+ T cells via an interaction with the αβ T-cell receptor. In humans, there are several MHC haplotypes, such as, for example, HLA-A2, HLA-A1, HLA-A3, HLA-A24, HLA-A28, HLA-A31, HLA-A33, HLA-A34, HLA-B7, HLA-B45 and HLA-Cw8, their sequences can be found at the kabbat data base, at www(dot)immuno(dot)bme(dot)nwu(dot)edu. Further information concerning MHC haplotypes can be found in Paul, B. Fundamental Immunology Lippincott-Raven Press.

The choice of tg-TCR depends upon the type and number of antigens that define the MHC-peptide complex of a target cell. For example, the tg-TCR may be chosen to recognize an MHC-peptide complex on a target cell associated with a particular disease state. Thus, for example, markers that may act as antigens for recognition by the tg-TCR may include those associated with viral, bacterial and parasitic infections and cancer cells. Examples are provided below.

To generate a successful tg-TCR, an appropriate target sequence needs to first be identified. Accordingly, a TCR may be isolated from an antigen reactive T cell (e.g. tumor reactive T cell) or, where this is not possible, alternative technologies can be employed. According to an exemplary embodiment, a transgenic animal (e.g. rabbit or mouse, preferably a human-HLA transgenic mouse) is immunized with human antigen peptides (e.g. tumor or viral antigens) to generate T cells expressing TCRs against the human antigens [as described e.g. in Stanislawski et al., Nat Immunol. (2001) 2(10):962-70]. According to another exemplary embodiment, antigen-specific T cells (e.g. tumor specific T cells) are isolated from a patient experiencing disease (e.g. tumor) remission and the reactive TCR sequences are isolated therefrom [as described e.g. in de Witte et al., Blood (2006) 108(3):870].

According to another exemplary embodiment, in vitro technologies are employed to alter the sequence of an existing TCR to enhance the avidity of a weakly reactive antigen-specific TCR with a target antigen (such methods are described below).

According to one embodiment, the tg-TCR of the invention is selected to recognize the antigen peptide-HLA complex with high avidity (i.e. the physical strength of the monomeric interaction between the TCR and a peptide-MHC-complex).

Producing cells with high functional avidity (i.e. that which effectively respond to antigens) can be achieved using any method known to one of ordinary skill in the art. Thus, according to one example, increasing the avidity of the tg-TCR is attained by increasing the affinity (i.e. strength of binding of a TCR to its ligand) of the tg-TCR or increasing the expression of the tg-TCR on the cell surface. According to one exemplary embodiment, increasing the TCR affinity is carried out by modification of tg-TCR genes. For example, one possible modification of the tg-TCR genes includes modifications to a complementarity determining region (CDR), e.g. third CDR (CDR3), of the tg-TCR. Accordingly, single or dual amino acid substitutions in the CDR chains (e.g. α or β chains) may be utilized in order to increase affinity of the tg-TCR and to enhance antigen-specific reactivity in transduced cells. According to another exemplary embodiment, increasing the functional avidity of tg-TCR is carried out by the removal of defined N-glycosylation motifs in the constant domains of tg-TCR chains. According to another exemplary embodiment, increasing the affinity is carried out by codon optimization.

Accordingly, rare codons of the tg-TCR are replaced by codons most frequently distributed in highly expressed human genes. During the optimization process cis-acting AT or GC rich sequence stretches, cryptic splicing and RNA instability motifs may also be removed. For further information, see e.g. Zhang and Morgan, Adv Drug Deliv Rev. (2012), supra, incorporated herein by reference.

According to one embodiment, the signaling module of the tg-TCR may comprise a single subunit or a plurality of signaling units. Accordingly, the tg-TCR of the invention may use co-receptors that act in concert with a TCR to initiate signal transduction following antigen receptor engagement, as well as any derivative or variant of thereof having the same functional capability.

According to one embodiment, the TCR signaling module comprises the CD3 complex (e.g. CD3 chains, e.g. CD3δ/ε, CD3γ/ε and/or zeta chains, e.g. ζ/ζ or ζ/η).

Additionally or alternatively, the TCR signaling module may comprise co-stimulatory protein receptors to provide additional signals to the T cell. These are discussed in detail for CAR molecules hereinbelow.

According to one embodiment, the tg-TCR may comprise a transmembrane domain as described in detail for CAR molecules hereinbelow.

Methods of transducing a cell with a TCR are described in detail hereinbelow.

As used herein the phrase “chimeric antigen receptor (CAR)” refers to a recombinant or synthetic molecule which combines specificity for a desired antigen with a T cell receptor-activating intracellular domain (i.e. T cell receptor signaling module) to generate a chimeric protein that exhibits cellular immune activity to the specific antigen. Typically, a CAR recognizes an antigen (e.g. protein or non-protein) expressed on the cell surface (rather than internal antigens) independently of the major histocompatibility complex (MHC).

Thus, the CAR of the invention generally comprises an extracellular domain comprising an antigen binding moiety, a transmembrane domain and an intracellular domain (i.e. the cytoplasmic domain) that is required for an efficient response of the T cell to the antigen.

### Antigen Binding Moiety

In one embodiment, the CAR of the invention comprises a target-specific binding element otherwise referred to as an antigen binding moiety. The choice of moiety depends upon the type and number of ligands (i.e. antigens) that define the surface of a target cell. For example, the antigen binding domain may be chosen to recognize a ligand (i.e. antigen) that acts as a cell surface marker on target cells associated with a particular disease state. Thus examples of cell surface markers that may act as ligands for the antigen moiety domain in the CAR of the invention include those associated with viral, bacterial and parasitic infections and cancer cells.

According to some embodiments of the invention, the antigen binding moiety comprises complementarity determining regions (CDRs) which are capable of specifically binding the antigen. Such CDRs can be obtained from an antibody.

The term “antibody” as used in this invention includes intact molecules as well as functional fragments thereof, such as Fab, Fab′, F(ab′)2, Fv, linear antibodies, scFv antibodies, and multispecific antibodies formed from antibody fragments that are capable of binding to the antigen. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; (5) Single chain antibody (“SCA”), a genetically engineered molecule containing the variable region of the light chain and the variable region of the heavy chain, linked by a suitable polypeptide linker as a genetically fused single chain molecule; (6) CDR peptide is a peptide coding for a single complementarity-determining region (CDR); and (7) Single domain antibodies (also called nanobodies), a genetically engineered single monomeric variable antibody domain which selectively binds to a specific antigen. Nanobodies have a molecular weight of only 12-15 kDa, which is much smaller than a common antibody (150-160 kDa).

An “antibody heavy chain,” as used herein, refers to the larger of the two types of polypeptide chains present in all antibody molecules in their naturally occurring conformation.

An “antibody light chain,” as used herein, refers to the smaller of the two types of polypeptide chains present in all antibody molecules in their naturally occurring conformations. Kappa- and lambda-light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat′l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Once the CDRs of an antibody are identified, using conventional genetic engineering techniques, expressible polynucleotides encoding any of the forms or fragments of antibodies described herein can be synthesized and modified in one of many ways in order to produce a spectrum of related-products.

According to some embodiments of the invention, the CDRs are derived from αβ T cell receptor (TCR) which specifically binds to the antigen.

According to some embodiments of the invention, the CDRs are derived from γδ T cell receptor (TCR) which specifically binds to the antigen.

According to some embodiments of the invention, the CDRs are derived from an engineered affinity-enhanced αβ T cell receptor or γδ T cell receptor (TCR) which specifically binds to the antigen (as discussed in detail herein above).

According to some embodiments of the invention, the CDRs are derived from an engineered αβ T cell receptor or γδ T cell receptor (TCR) with improved stability or any other biophysical property.

According to some embodiments of the invention, the CDRs are derived from a T cell receptor-like (TCRLs) antibody which specifically binds to the antigen. Examples of TCRLs and methods of generating same are described in WO03/068201, WO2008/120203, WO2012/007950, WO2009125395, WO2009/125394, each of which is fully incorporated herein by their entirety.

According to some embodiments of the invention, the antigen binding domain comprises a single chain Fv (scFv) molecule.

According to specific embodiments of the invention, the scFv molecule targets HER2, such as, for example, the scFv molecule derived from N29 monoclonal antibody as discussed in Li et al. Cancer Gene Ther (2008) 15(6):382-92, incorporated herein by reference.

According to specific embodiments of the invention, the scFv molecule targets CD19 (e.g. is an antigen binding domain from FMC63) or CD20 (e.g. is an antigen binding domain from Leu16) as discussed in Schneider et al. Journal for ImmunoTherapy of Cancer (2017) 5:42, incorporated herein by reference.

According to specific embodiments of the invention, the scFv molecule targets BCMA.

### Cytoplasmic Domain

The cytoplasmic domain (also referred to as “intracellular signaling domain” or “T cell receptor signaling module”) of the CAR molecule of the invention is responsible for activation of at least one of the normal effector functions of the cell in which the CAR has been placed in.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Preferred examples of intracellular signaling domains for use in the CAR molecule of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs (ITAMs).

Examples of ITAM containing primary cytoplasmic signaling sequences that are of particular use in the invention include those derived from TCR zeta, FcR gamma, FcR beta, CD3 gamma, CD3 delta, CD3 epsilon, CD5, CD22, CD79a, CD79b, and CD66d. It is particularly preferred that cytoplasmic signaling molecule in the CAR of the invention comprises a cytoplasmic signaling sequence derived from CD3 zeta.

In a preferred embodiment, the cytoplasmic domain of the CAR can be designed to comprise the CD3-zeta signaling domain by itself or combined with any other desired cytoplasmic domain(s) useful in the context of the CAR of the invention. For example, the cytoplasmic domain of the CAR can comprise a CD3 zeta chain portion and a co-stimulatory signaling region. The co-stimulatory signaling region refers to a portion of the CAR comprising the intracellular domain of a co-stimulatory molecule. A co-stimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like.

According to some embodiments of the invention, the intracellular domain comprises a co-stimulatory signaling region and a zeta chain portion. The co-stimulatory signaling region refers to a portion of the CAR molecule comprising the intracellular domain of a co-stimulatory molecule. Co-stimulatory molecules are cell surface molecules other than antigen receptors or their ligands that are required for an efficient response of lymphocytes to antigen.

“Co-stimulatory ligand,” as the term is used herein, includes a molecule on an antigen presenting cell [e.g., an aAPC (artificial antigen presenting cell), dendritic cell, B cell, and the like] that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible co-stimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or down regulation of key molecules.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter cilia, a MHC Class I molecule loaded with a peptide, an anti-CD3 antibody, a superagonist anti-CD28 antibody, and a superagonist anti-CD2 antibody.

With respect to the cytoplasmic domain, the CAR molecule of some embodiments of the invention can be designed to comprise the CD28 and/or 4-1BB signaling domain by itself or be combined with any other desired cytoplasmic domain(s) useful in the context of the CAR molecule of some embodiments of the invention. In one embodiment, the cytoplasmic domain of the CAR can be designed to further comprise the signaling domain of CD3-zeta. For example, the cytoplasmic domain of the CAR can include, but is not limited to, CD3-zeta, 4-1BB and CD28 signaling modules and combinations thereof.

According to some embodiments of the invention, the intracellular domain comprises at least one, e.g., at least two, at least three, at least four, at least five, e.g., at least six of the polypeptides selected from the group consisting of: CD3ζ (CD247, CD3z), FcR-γ, CD27, CD28, 4-1BB/CD137, ICOS, OX40/CD134, DAP10, tumor necrosis factor receptor (TNFr) and Lsk.

According to some embodiments of the invention, the intracellular domain comprises the CD3ζ-chain [CD247 molecule, also known as “CD3-ZETA” and “CD3z”; GenBank Accession NOs. NP_000725.1 and NP_932170.1], which is the primary transmitter of signals from endogenous TCRs.

According to some embodiments of the invention, the intracellular domain comprises various co-stimulatory protein receptors to the cytoplasmic tail of the CAR to provide additional signals to the T cell (“second generation” CAR). Examples include, but are not limited to, CD28 [e.g., GenBank Accession Nos. NP_001230006.1, NP_001230007.1, NP_006130.1], 4-1BB [tumor necrosis factor receptor superfamily, member 9 (TNFRSF9), also known as “CD137”, e.g., GenBank Accession No. NP_001552.2], ICOS [inducible T-cell co-stimulator, e.g., GenBank Accession No. NP_036224.1], DAP10 [hematopoietic cell signal transducer, e.g., GenBank Accession Nos. NP_001007470, NP_055081.1] and Lsk [LCK proto-oncogene, Src family tyrosine kinase, e.g., GenBank Accession Nos. NP_001036236.1, NP_005347.3]. Preclinical studies have indicated that the “second generation of CAR designs improves the antitumor activity of T cells.

According to some embodiments of the invention, the intracellular domain comprises multiple signaling domains, such as CD3z-CD28-4-1BB or CD3z-CD28-OX40, to further augment potency. The term “OX40” refers to the tumor necrosis factor receptor superfamily, member 4 (TNFRSF4), e.g., GenBank Accession No. NP_003318.1 (“third-generation” CARs).

According to some embodiments of the invention, the intracellular domain comprises CD28-CD3z, CD3z, CD28-CD137-CD3z. The term “CD137” refers to tumor necrosis factor receptor superfamily, member 9 (TNFRSF9), e.g., GenBank Accession No. NP_001552.2.

According to some embodiments of the invention, the intracellular domain comprises CD3z, CD28 and a tumor necrosis factor receptor (TNFr).

According to some embodiments of the invention, the CAR comprises a CD3 zeta chain.

According to some embodiments of the invention, the CAR comprises at least one co-stimulatory domain selected from the group consisting of CD28, CD134/OX40, CD137/4-1BB, Lck, ICOS and DAP10.

According to some embodiments of the invention, the CAR comprises at least two co-stimulatory domains selected from the group consisting of CD28, CD134/OX40, CD137/4-1BB, Lck, ICOS and DAP10.

According to a specific embodiment of the invention, the CAR comprises a FcR gamma chain (e.g. a γITAM subunit domain).

According to a specific embodiment of the invention, the CAR comprises a CD28 co-stimulation domain.

According to a specific embodiment of the invention, the CAR comprises a CD28 co-stimulation domain and the γITAM subunit domain.

### Transmembrane Domain

The transmembrane domain of the CAR may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain.

According to a specific embodiment, the transmembrane domain comprises CD8.

According to a specific embodiment, the transmembrane domain comprises CD28.

Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

According to some embodiments of the invention, the transmembrane domain comprised in the CAR molecule of some embodiments of the invention is a transmembrane domain that is naturally associated with one of the domains in the CAR. According to some embodiments of the invention, the transmembrane domain can be selected or modified by amino acid substitution to avoid binding of such domains to the transmembrane domains of the same or different surface membrane proteins to minimize interactions with other members of the receptor complex.

According to some embodiments of the invention, the transmembrane domain is the CD8α hinge domain.

According to some embodiments, between the extracellular domain and the transmembrane domain of the CAR molecule, or between the cytoplasmic domain and the transmembrane domain of the CAR molecule, there may be incorporated a spacer domain. As used herein, the term “spacer domain” generally means any oligo- or polypeptide that functions to link the transmembrane domain to, either the extracellular domain or, the cytoplasmic domain in the polypeptide chain. A spacer domain may comprise up to 300 amino acids, preferably 10 to 100 amino acids and most preferably 25 to 50 amino acids.

According to a specific embodiment, the CAR comprises the subunits scFv-4-1BB-CD3ζ.

According to a specific embodiment, the CAR comprises the subunits scFv-CD28-CD3ζ.

According to a specific embodiment, the CAR comprises the subunits scFv-CD28-γITAM.

According to a specific embodiment, the CAR comprises the subunits scFv (N29)-CD28-CD3ζ.

According to a specific embodiment, the CAR comprises the subunits scFv (N29)-CD28-γITAM.

Exemplary CAR vectors which may be used according to one embodiment of the invention include, but are not limited to, those utilizing the antigen binding domains from FMC63 (anti-CD19) and Leu16 (anti-CD20) antibodies [discussed in Schneider et al., Journal for ImmunoTherapy of Cancer (2017) 5:42, incorporated herein by reference]. Additional exemplary CAR vectors include e.g. MUC1-CAR-T, NKG2D-CAR-T, BCMA-CAR-T, EGFRvIII-CAR-T, GD2 CAR-T and anti-CD133 CAR-T cells (CART133) discussed in Guo et al., Cancers (2021) 13: 1955, incorporated herein by reference.

Commercially available vectors may also be used in accordance with the present invention, such as but not limited to, pCDCAR1 CD19 h(28ζ) (Cat. No. CAR-YF026), pCDCAR1 CD19 h(BBζ) (Cat. No. CAR-YF025), pCDCAR1 CD19 h(BBζ) (Cat. No. CAR-YF090), pCDCAR1 HER2 h(28BBζ) (Cat. No. CAR-LC252), pCDCAR1 HER2 h(28BBζ) (Cat. No. CAR-LC188), pCDCAR1 HER2 h(28BBζ) (Cat. No. CAR-LC253), pCDCAR1 BCMA h(BBζ) (Cat. No. CAR-LC002), pCDCAR1 BCMA h(BBζ) (Cat. No. CAR-LC135) and pCDCAR1 BCMA h(BBζ) (Cat. No. CAR-LC138), all available from Creative Biolabs, USA.

Combo CAR vectors may also be used in accordance with the present invention, such as but not limited to, anti-CD19 and anti-CD22 CAR-T cells (CAR19/22 T cell cocktail) and CAR-T with CD19 and CD123 dual expression, discussed in Guo et al., Cancers (2021) 13: 1955, incorporated herein by reference.

As mentioned, the cell surface receptor of the cell of the invention (e.g. tg-TCR and/or CAR) binds to an antigen (e.g. on a target cell).

According to one embodiment, the cell surface receptor of the cell of the invention (e.g. tg-TCR and/or CAR) binds to more than one antigen (e.g. on a target cell).

According to one embodiment, the cell surface receptor of the cell of the invention (e.g. tg-TCR and/or CAR) is bispecific (e.g. targets two distinct antigens, e.g. CD33 and CLL1).

According to one embodiment, the antigen may comprise a tumor associated antigen, a viral antigen, a bacterial antigen, a fungal antigen, a protozoa antigen, and/or a parasite antigen.

As used herein the phrase “tumor antigen” refers to an antigen that is common to specific hyperproliferative disorders such as cancer. Tumor antigens are proteins that are produced by tumor cells that elicit an immune response, particularly T-cell mediated immune responses. The selection of the antigen binding moiety of the invention will depend on the particular type of cancer to be treated.

According to one embodiment, the tumor antigen is associated with a solid tumor.

According to one embodiment, the tumor antigen is associated with a hematologic malignancy.

The type of tumor antigen referred to in the invention includes a tumor-specific antigen (TSA) or a tumor-associated antigen (TAA). A “TSA” refers to a protein or polypeptide antigen unique to tumor cells and which does not occur on other cells in the body. A “TAA” refers to a protein or polypeptide antigen that is expressed by a tumor cell. For example, a TAA may be one or more surface proteins or polypeptides, nuclear proteins or glycoproteins, or fragments thereof, of a tumor cell.

The antigens discussed herein are merely included by way of example. The list is not intended to be exclusive and further examples will be readily apparent to those of skill in the art.

Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER2, CD19, CD20, idiotype) have been used as targets for passive immunotherapy with monoclonal antibodies with limited success.

Non-limiting examples of TSA or TAA antigens include the following: Differentiation antigens such as MART-1/MelanA (MART-1), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.291\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

Further examples of tumor antigens include, but are not limited to, A33, BAGE, Bcl-2, β-catenin, BCMA, CA125, CA19-9, CD5, CD19, CD20, CD21, CD22, CD33/IL3Ra, CD34, CD37, CD45, CD123, CD135 (FLT3), CD138, carcinoembryonic antigen (CEA), CLL1, c-Met, CS-1, cyclin B1, DAGE, EBNA, EGFR, EGFRvIII, ephrinB2, estrogen receptor, FAP, ferritin, folate-binding protein, GAGE, G250, GD-2, GM2, gp75, gp100 (Pmel 17), Glycolipid F77, HER2/neu, HPV E6, HPV E7, Ki-67, LRP, mesothelin, MY-ESO-1, MART-1, MAGE A3, p53, PRAME, PR1, PSMA, ROR1, SLAMF7, WT1 (wilms tumor), and the like. Further tumor antigens are provided in van der Bruggen P, Stroobant V, Vigneron N, Van den Eynde B. Peptide database: T cell-defined tumor antigens. Cancer Immun (2013), www(dot)cancerimmunity(dot)org/peptide/, incorporated herein by reference.

Following is a list of tumor antigens which may be used according to the teachings of some embodiments of the invention.

TABLE 1A

list of tumor antigens recognized by tg-TCR

| | | | |
|---|---|---|---|
| Cancer | TAA/Marker | Exemplary GenBank Accession No. of tumor antigens | HLA |
| Transitional cell carcinoma | Uroplakin II (UPKII) | NP_006751.1 | HLA-A2 |
| Transitional cell carcinoma | Uroplakin Ia (UPK1A) | NP_001268372.1; NP_008931.1 | HLA-A2 |
| Carcinoma of the prostate | prostate specific antigen (NPSA) | AAO16090.1 | HLA-A2 |
| Carcinoma of the prostate | prostate specific membrane antigen (PSCA) | NP_005663.2 | HLA-A2 |
| Carcinoma of the prostate | prostate acid phosphatase (ACPP) | NP_001090.2; NP_001127666.1; NP_001278966.1 | HLA-A2 |
| Breast cancer | BA-46 MFGE8 milk fat globule-EGF factor 8 protein [lactadherin] | NP_001108086.1; NP_005919.2; | HLA-A2 |
| Breast cancer | Mucin 1 (MUC1) | NP_001018016.1; NP_001018017.1; NP_001037855.1; NP_001037856.1; NP_001037857.1; NP_001037858.1; NP_001191214.1; NP_001191215.1; NP_001191216.1; NP_001191217.1; NP_001191218.1; NP_001191219.1; NP_001191220.1; NP_001191221.1; NP_001191222.1; NP_001191223.1; NP_001191224.1; NP_001191225.1; NP_001191226.1; NP_002447.4 | HLA-A2 |
| Melanoma | premelanosome protein (PMEL; also known as Gp100) | NP_001186982.1; NP_001186983.1; NP_008859.1 | HLA-A2 |
| Melanoma | melan-A (MLANA; also known as MART-1) | NP_005502.1; | HLA-A2 |
| All tumors | telomerase reverse transcriptase (TERT) | NP_001180305.1; NP_937983.2 | HLA-A2 |
| Leukemia and Burkitts Lymphoma | TAX tax p40 [Human T-lymphotropic virus 1] and Tax [Human T-lymphotropic virus 4]; | NP_057864.1; YP_002455788.1 | HLA-A2 |
| Carcinomas | NY-ESO cancer/testis antigen 1B (CTAG1B) | NP_001318.1 | HLA-A2 |
| Melanoma | Melanoma antigen family A1 (MAGEA1) | NP_004979.3 | HLA-A2 |
| Melanoma Synovial cell cancer Esophageal cancer Myeloma | Melanoma antigen family A3 (MAGEA3, MAGE-A3) | NP_005353.1 | HLA-A24 |
| Carcinomas | HER2; erb-b2 receptor tyrosine kinase 2 (ERBB2) | NP_001005862.1; NP_001276865.1; NP_001276866.1; NP_001276867.1; NP_004439.2; | HLA-A2 |
| Melanoma | Beta-catenine; catenin (cadherin-associated protein), beta 1, 88 kDa (CTNNB1) | NP_001091679.1; NP_001091680.1; NP_001895.1; | HLA-A24 |
| Melanoma | Tyrosinase (TYR) | NP_000363.1 | HLA-DRB1 |
| Leukemia | Bcr-abl | AAA35594.1 | HLA-A2 |
| Head and neck | caspase 8, apoptosis-related cysteine peptidase (CASP8) | NP_001073593.1; NP_001073594.1; NP_001219.2; NP_203519.1; NP_203520.1; NP_203522.1 | HLA-B35 |
| Colorectal cancer | Carcinoembryonic antigen (CEA) | NP_001278413.1, NP_001295327.1, NP_004354.3 | |
| Melanoma Synovial cell cancer | NY-ESO-1 | NP_640343 NP_001318 | |
| Myeloid leukemia | PR1 | NP_195097.1 | HLA-A2 |

TABLE 1B

- list of CAR targets for hematological malignancies

| | | |
|---|---|---|
| Antigen | Disease | Exemplary GenBank Accession No. of the antigens |
| CD19 | B-cell malignancy | NP_001171569, NP_001761.3 |
| CD20 | B-cell malignancy | NP_068769.2, NP_690605.1 |
| CD22 | B-cell malignancy | NP_001172028.1, NP_001172029.1, NP_001172030.1, NP_001265346.1, NP_001762.2 |
| CD30/ Tumor necrosis factor receptor superfamily member 8 (TNFRSF8) | B-cell malignancy | NP_001234.3, NP_001268359.2 |
| CD33 | Myeloid malignancy e.g. Acute myeloid leukemia (AML) | NP_001076087.1, NP_001171079.1, NP_001763.3, XP_011525834.1 |
| CD37 | B cell lymphoma | NP_001035120.1, NP_001765.1, XP_011525846.1, XP_016883002.1 |
| CD70 | B-cell/T-cell malignancy | NP_001243.1, NP_001317261.1 |
| CD123/ Interleukin-3 receptor subunit alpha (IL3RA) | Myeloid malignancy e.g. Acute myeloid leukemia (AML) | NP_001254642.1, NP_002174.1, XP_005274488.1, XP_005274837.1 |
| CD135 (FLT3) | Acute myeloid leukemia (AML) | NP_004110.2 |
| Kappa | B-cell malignancy | |
| Lewis Y | Myeloid malignancy | |
| NKG2D ligands | Myeloid malignancy | NP_001186734.1, NP_031386.2 |
| ROR1 | B-cell malignancy | NP_001077061.1, NP_005003.2 |
| BCMA/Tumor necrosis factor receptor superfamily member 17 (TNFRSF17) | Multiple myeloma | NP_001183.2 |
| SLAMF7 | Multiple myeloma | NP_001269517.1, NP_001269518.1, NP_001269519.1, NP_001269520.1, NP_001269521.1, NP_001269524.1, NP_067004.3 |
| CLL1/ CLEC12A | Acute myeloid leukemia (AML) | NP_001193939.1, NP_001287659.1, NP_612210.4, NP_963917.2 |

(adapted from Dotti et al., Immunol Rev. (2014) 257(1): . doi:10.1111/imr.12131)

TABLE 1C

list of CAR targets for solid tumors

| | | |
|---|---|---|
| Antigen | Disease | Exemplary GenBank Accession No. of the antigens |
| B7H3/ CD276 | sarcoma, glioma | NP_001019907.1, NP_001316557.1, NP_001316558.1, NP_079516.1 |
| Carbonic anhydrase IX (CAIX) | kidney | NP_001207.2 |
| CD44 v6/v7 | cervical | |
| CD171/NCAM-L1 | neuroblastoma,, colorectal | NP_000416.1, NP_001137435.1, NP_001265045.1, NP_076493.1, |
| Carcinoembryonic antigen (CEA) | colon | NP_001278413.1, NP_001295327.1, NP_004354.3 |
| Epidermal growth factor receptor variant III (EGFRvIII ) | glioma | NP_005219.2, NP_958439.1, NP_958440.1, NP_958441.1 |
| Epithelial Glycoprotein 2 (EGP-2)/ Ep-CAM/ Epithelial Glycoprotein 40 (EGP-40) | carcinomas, colon | NP_002345.2 |
| Ephrin type-A receptor 2 (EphA2) | glioma, lung | NP_004422.2 |
| ErbB2 (HER2) | breast, lung, prostate, glioma, gastric, gastroesophageal, oesophageal, ovarian, endometrial, urothelial and bladder | NP_001005862.1, NP_001276865.1, NP_001276867.1, NP_004439.2 |
| ErbB3 (HER3) | breast, ovarian | NP_001005915.1, NP_001973.2 |
| ErbB4 (HER4) | breast, ovarian | NP_001036064.1, NP_005226.1 |
| HLA-A1/MAGE-1 (Melanoma-associated antigen 1) | melanoma | NP_004979.3 |
| HLA-A2/NY-ESO-1 (New York esophageal squamous cell carcinoma 1) | sarcoma, melanoma | NP_001318.1, NP_640343.1 |
| Folate receptor alpha (FR-α) | ovarian | NP_000793.1, NP_057936.1, NP_057937.1, NP_057941.1 |
| FAP^{∗∗} | cancer associated fibroblasts | NP_001278736.1, NP_004451.2 |
| GD2 (Ganglioside G2) | neuroblastoma, sarcoma, melanoma | |
| GD3 (Ganglioside G3) | melanoma, lung cancer | |
| HMW-MAA (high molecular weight melanoma-associated antigen)/ CSPG4 | melanoma | NP_001888.2 |
| IL11Rα (interleukin-11 receptor subunit alpha) | osteosarcoma | NP_001136256.1 |
| IL13Rα2 (Interleukin-13 receptor subunit alpha-2) | glioma | NP_000631.1 |
| Lewis Y (FUT3/FUT2) | breast/ovarian/pancreatic | NP_000140.1, NP_001091108.1, NP_001091109.1, NP_001091110.1 |
| L1-cell adhesion molecule (L1CAM) | neuroblastoma | NP_000416.1, NP_001137435.1, NP_001265045.1, NP_076493.1 |
| Mesothelin | mesothelioma, breast, pancreas | NP_001170826.1, NP_005814.2, NP_037536.2 |
| Mucin 1 (MUC1) | ovarian, breast, prostate | NP_001018016.1; NP_001018017.1; NP_001037855.1; NP_001037856.1; NP_001037857.1; NP_001037858.1; NP_001191214.1; NP_001191215.1; NP_001191216.1; NP_001191217.1; NP_001191218.1; NP_001191219.1; NP_001191220.1; NP_001191221.1; NP_001191222.1; NP_001191223.1; NP_001191224.1; NP_001191225.1; NP_001191226.1; NP_002447.4 |
| NKG2D ligands | ovarian, sacoma | NP_001186734.1, NP_031386.2 |
| PSCA (Prostate stem cell antigen) | prostate, pancreatic | NP_005663.2 |
| PSMA (Prostate-specific membrane antigen) | prostate | NP_001014986.1, NP_001180400.1, NP_001180401.1, NP_001180402.1, NP_004467.1 |
| TAG-72 (Tumor-associated glycoprotein 72) | colon | |
| VEGFR-2^{∗∗} | tumor vasculature | NP_002244.1 |

Of note, many antigens are expressed on several malignancies, only examples are listed
^{∗∗} marks expression on the tumor stroma
(adapted from Dotti et al., Immunol Rev. (2014) 257(1): . doi:10.1111/imr.12131)

Additional CAR targets for solid tumors are described in Ma et al., Int. J. Biol. Sci. (2019) 15(12): 2548-2560, incorporated herein by reference.

According to a specific embodiment, the target antigen is HER2.

According to a specific embodiment, the target antigen is CD19.

According to a specific embodiment, the target antigen is BCMA.

According to some embodiments of the invention, the cell surface receptor of the cell of the invention (e.g. tg-TCR and/or CAR) binds to a viral antigen (e.g. on a target cell).

According to some embodiments of the invention, the viral antigen may be derived from any virus, such as but not limited to, human immunodeficiency virus (HIV), influenza, Cytomegalovirus (CMV), T-cell leukemia virus type 1 (TAX), hepatitis C virus (HCV), (HBV), Epstein-Barr virus (EBV), Adenovirus (Adv), cold viruses, flu viruses, hepatitis A, B, and C viruses, herpes simplex, Japanese encephalitis, measles, polio, rabies, respiratory syncytial, rubella, smallpox, varicella zoster, rotavirus, West Nile virus, Polyomavirus (e.g. BK virus), severe acute respiratory syndrome (SARS) e.g. severe acute respiratory syndrome coronavirus 2 (SARS-CoV-2), and/or zika virus.

According to some embodiments of the invention, the viral antigens include, but are not limited to, viral epitopes from a polypeptide selected from the group consisting of: human T cell lymphotropic virus type I (HTLV-1) transcription factor (TAX), influenza matrix protein epitope, Epstein-Bar virus (EBV)-derived epitope, HIV-1 RT, HIV Gag, HIV Pol, influenza membrane protein M1, influenza hemagglutinin, influenza neuraminidase, influenza nucleoprotein, influenza nucleoprotein, influenza matrix protein (M 1), influenza ion channel (M2), influenza non-structural protein NS-1, influenza non-structural protein NS-2, influenza PA, influenza PB1, influenza PB2, influenza BM2 protein, influenza NB protein, influenza nucleocapsid protein, Cytomegalovirus (CMV) phosphorylated matrix protein (pp65), TAX, hepatitis C virus (HCV), HBV pre-S protein 85-66, HTLV-1 tax 11-19, HBV surface antigen 185-194, Severe acute respiratory syndrome (SARS-CoV) protein S1, SARS-CoV protein RBD, SARS-CoV Nuclecapsid protein, SARS-CoV protein Plpro, Severe acute respiratory syndrome coronavirus 2 (SARS-CoV-2) protein S1, SARS-CoV-2 protein S2, SARS-CoV-2 protein S1+S2 ECD, SARS-CoV-2 protein RBD, SARS-CoV-2 protein N antigen, SARS-CoV-2 protein S antigen or SARS-CoV-2 nuclecapsid protein.

According to some embodiments of the invention, the cell surface receptor of the cell of the invention (e.g. tg-TCR and/or CAR) binds to a bacterial antigen (e.g. on a target cell).

According to some embodiments of the invention, the bacterial antigen may be derived from any bacteria, such as but not limited to, anthrax; gram-negative bacilli, chlamydia, diptheria, haemophilus influenza, Helicobacter pylori, malaria, Mycobacterium tuberculosis, pertussis toxin, pneumococcus, rickettsiae, staphylococcus, streptococcus and tetanus.

According to some embodiments of the invention, the bacterial antigens include, but are not limited to, anthrax antigens include, but are not limited to, anthrax protective antigen; gram-negative bacilli antigens include, but are not limited to, lipopolysaccharides; haemophilus influenza antigens include, but are not limited to, capsular polysaccharides; diptheria antigens include, but are not limited to, diptheria toxin; Mycobacterium tuberculosis antigens include, but are not limited to, mycolic acid, heat shock protein 65 (HSP65), the 30 kDa major secreted protein and antigen 85A; pertussis toxin antigens include, but are not limited to, hemagglutinin, pertactin, FIM2, FIM3 and adenylate cyclase; pneumococcal antigens include, but are not limited to, pneumolysin and pneumococcal capsular polysaccharides; rickettsiae antigens include, but are not limited to, rompA; streptococcal antigens include, but are not limited to, M proteins; and tetanus antigens include, but are not limited to, tetanus toxin.

According to some embodiments of the invention, the antigen is a superbug antigen (e.g. multi-drug resistant bacteria). Examples of superbugs include, but are not limited to, Enterococcus faecium, Clostridium difficile, Acinetobacter baumannii, Pseudomonas aeruginosa, and Enterobacteriaceae (including Escherichia coli, Klebsiella pneumoniae, Enterobacter spp.).

According to some embodiments of the invention, the cell surface receptor of the cell of the invention (e.g. tg-TCR and/or CAR) binds to a fungal antigen (e.g. on a target cell). Examples of fungi include, but are not limited to, candida, coccidiodes, cryptococcus, histoplasma, leishmania, plasmodium, protozoa, parasites, schistosomae, tinea, toxoplasma, and trypanosoma cruzi.

According to some embodiments of the invention, the fungal antigens include, but are not limited to, coccidiodes antigens include, but are not limited to, spherule antigens; cryptococcal antigens include, but are not limited to, capsular polysaccharides; histoplasma antigens include, but are not limited to, heat shock protein 60 (HSP60); leishmania antigens include, but are not limited to, gp63 and lipophosphoglycan; plasmodium falciparum antigens include, but are not limited to, merozoite surface antigens, sporozoite surface antigens, circumsporozoite antigens, gametocyte/gamete surface antigens, protozoal and other parasitic antigens including the blood-stage antigen pf 155/RESA; schistosomae antigens include, but are not limited to, glutathione-S-transferase and paramyosin; tinea fungal antigens include, but are not limited to, trichophytin; toxoplasma antigens include, but are not limited to, SAG-1 and p30; and trypanosoma cruzi antigens include, but are not limited to, the 75-77 kDa antigen and the 56 kDa antigen.

According to some embodiments of the invention there is provided a method of generating the genetically modified veto cell having the Tcm phenotype.

According to some embodiments of the invention, the method comprises: (a) providing a population of cells comprising T cells, the T cells comprising at least 40% memory CD8⁺ T cells; and (b) culturing the population of cells comprising T cells with an antigen or antigens under conditions which allow enrichment of tolerance-inducing antigen-specific cells having a Tcm phenotype, the cells being non-GVH inducing cells.

According to some embodiments of the invention, the method comprises: (a) treating peripheral blood mononuclear cells (PBMCs) with an agent capable of depleting CD4⁺, CD56⁺ and CD45RA⁺ cells, or with an agent capable of selecting CD45RO⁺, CD8⁺ cells, so as to obtain a population of cells comprising T cells enriched of memory CD8⁺ T cells comprising a CD45RO⁺CD45RA⁻CD8⁺ phenotype; and (b) culturing the population of cells comprising T cells with an antigen or antigens under conditions which allow enrichment of tolerance-inducing antigen-specific cells having a central memory T-lymphocyte (Tcm) phenotype, the cells being non-GVH inducing cells.

The term “a population of cells comprising T cells” refers to a heterogeneous mixture of lymphocytes which typically comprises T cells having numerous signatures, functions and capable of binding various antigens (e.g. cytotoxic T cells, memory T cells, effector T cells etc.).

According to one embodiment, the population of cells is deprived of naïve T cells.

According to one embodiment, the population of cells comprises memory T cells.

According to some embodiments, other lymphocytes may be comprised in the population of cells, such as but not limited to, myeloid cells (e.g. monocytes) and B cells.

The term “memory CD8⁺ T cells” as used herein refers to a subset of T lymphocytes which have previously encountered and responded to an antigen, also referred to as antigen experienced T cells (also referred to herein as “memory T cells”).

According to one embodiment, the memory CD8⁺ T cells comprise at least about 20%, at least about 30%, at least about 40%, at least about 50%, at least about 60%, at least about 70%, at least about 80%, at least about 90%, at least about 95%, at least about 99%, or even 100% of the T cells in the population of cells.

According to one embodiment, the memory T cells comprise T cells expressing a CD8 marker (i.e. CD8⁺ T cells).

According to another embodiment, the memory T cells comprise a CD8⁺CD45RO⁺ phenotype.

According to another embodiment, the memory T cells comprise a CD8⁺CD45RA⁻ phenotype.

According to another embodiment, the memory T cells comprise a CD8⁺CD45RO⁺CD45RA⁻ phenotype.

According to one embodiment, the memory T cells are devoid of CD45RA⁺ cells.

According to one embodiment, the memory T cells are devoid of CD4⁺ and/or CD56⁺ cells.

Selection of memory CD8⁺ T cells may be affected by selection of cells co-expressing CD8⁺ and CD45RA⁻ and/or cells co-expressing CD8⁺ and CD45RO⁺ and may be carried out using any method known in the art, such as by affinity based purification (e.g. such as by the use of MACS beads, FACS sorter and/or capture ELISA labeling).

Selection of memory CD8⁺ T cells may be further affected by selection of effector T cells and central memory T cells, the latter expressing e.g. CD62L, CCR7, CD27 and/or CD28.

According to one embodiment, memory T cells are obtained from a lymphoid tissue, such as from lymph nodes or spleen.

According to one embodiment, memory T cells are obtained from peripheral blood mononuclear cells (PBMCs).

In order to obtain a cell population comprising a high purity of CD8⁺ memory T cells (e.g. at least about 40% CD8⁺ memory T cells out of the total T cell population) or in order to increase the number of CD8⁺ memory T cells, PBMCs may be depleted of naïve cells, e.g. CD45RA⁺ cells, may be depleted of adherent cells (e.g. monocytes, macrophages), may be depleted of CD4⁺ cells (e.g. T helper cells), may be depleted of CD56⁺ cells (e.g. NK cells) or may be depleted any other cells not comprising a memory T cell phenotype.

According to one embodiment, providing a population of cells comprising a high level of memory T cells (e.g. at least about 40% CD8⁺ memory T cells out of the total T cells) is affected by treating PBMCs with an agent capable of depleting CD4⁺, CD56⁺ and CD45RA⁺ cells, so as to obtain a population in which the T cells are enriched of CD8⁺ memory T cells comprising a CD45RO⁺CD45RA⁻CD8⁺ phenotype. Depletion cells from PBMCs, may be carried out using any method known in the art, such as by affinity based purification (e.g. such as by the use of MACS beads, FACS sorter and/or capture ELISA labeling).

According to one embodiment, providing a population of cells comprising a high level of CD8⁺ memory T cells (e.g. at least about 40% CD8⁺ memory T cells out of the total T cells) is affected by treating PBMCs with an agent capable of selecting CD45RO⁺, CD8⁺ cells, so as to obtain a population in which the T cells are enriched of CD8⁺ memory T cells comprising a CD45RO⁺CD45RA⁻CD8⁺ phenotype. Selection cells from PBMCs may be carried out using any method known in the art, such as by affinity based purification (e.g. such as by the use of MACS beads, FACS sorter and/or capture ELISA labeling).

Depletion of adherent cells from PBMCs may be carried out using any method known in the art, e.g. by culturing the PBMCs on a cell culture dish (e.g. for 2-6 hours) and collecting the non-adherent cells.

In order to deplete alloreactive clones from the memory T cell pool, the population of cells enriched of CD8⁺ memory T cells is cultured with an antigen or antigens under conditions which allow enrichment of tolerance-inducing antigen-specific cells having a Tcm phenotype.

For example, an antigen or antigens can be whole cells (e.g. live or dead cell/s), cell fraction/s (e.g. lysed cell/s), cell antigen/s (e.g. cell surface antigen/s), a protein extract, a purified protein or a synthetic peptide. For example, an antigen or antigens of some embodiment of the invention include antigen/s of viruses (i.e. viral antigens), antigen/s of bacteria (i.e. bacterial antigens), antigen/s of fungi (e.g. fungi antigens) or antigen/s associated with a malignant disease (e.g. tumor antigen/s).

According to one embodiment, the antigen or antigens used for depletion of alloreactivity (i.e. for generation of veto cells) is distinct from the antigen or antigens used for targeting by the CAR or the tg-TCR. Thus, for example, the antigen or antigens used for depletion of alloreactivity may comprise viral antigen or antigens, while the antigen or antigens used for targeting by the CAR or the tg-TCR comprise tumor antigen or antigens.

According to one embodiment, the antigen or antigens comprise third party antigen or antigens.

As used herein the phrase “third party antigen or antigens” refers to a soluble or non-soluble (such as membrane associated) antigen or antigens which are not an endogenous part of the donor or recipient, as depicted in detail infra.

According to an embodiment, the antigen or antigens is of an infectious organism (e.g., viral, bacterial, fungal organism) which typically affects immune comprised subjects, such as transplantation patients. Exemplary infectious organisms which may affect immune comprised patients include, but are not limited to, viruses such as parvovirus (e.g. parvovirus B19), rotavirus, varicella-zoster virus (VZV), Herpes simplex virus (HSV), cytomegalovirus (CMV), Epstein-Barr virus (EBV), Polyomavirus (e.g. BK virus); bacteria such as S pneumoniae, P aeruginosa, Legionella pneumophila, L monocytogenes, Nocardia species, Mycobacterium species, S aureus, Nocardia species, P aeruginosa, Serratia species, Chromobacterium, streptococci, Burkholderia, Mycobacterium (e.g. Mycobacterium avium-intracellulare complex), encapsulated bacteria such as S pneumoniae, H influenzae and N meningitidis; fungi such as P jiroveci, Candida, and Aspergillus; and parasites such as Toxoplasma species, cryptosporidia and Strongyloides species.

Exemplary viral, bacterial, fungal and superbug antigen/s which may be used to deplete alloreactive clones from the memory T cell pool are discussed above.

According to a specific embodiment, the antigen is a viral antigen, such as but not limited to, an Adenovirus antigen including, but not limited to, Adv-penton or Adv-hexon; a BK Virus antigen including, but not limited to, BKV LT; BKV (capsid VP1), BKV (capsid protein VP2), BKV (capsid protein VP2, isoporm VP3), BKV (small T antigen); a CMV antigen including, but not limited to, envelope glycoprotein B, CMV IE-1 and CMV pp65; an EBV antigen including, but not limited to, EBV LMP2, EBV BZLF1, EBV EBNA1, EBV P18, and EBV P23; a hepatitis antigen including, but not limited to, the S, M, and L proteins of hepatitis B virus, the pre-S antigen of hepatitis B virus, HBCAG DELTA, HBV HBE, hepatitis C viral RNA, HCV NS3 and HCV NS4; a herpes simplex viral antigen including, but not limited to, immediate early proteins and glycoprotein D; a HIV antigen including, but not limited to, gene products of the gag, pol, and env genes such as HIV gp32, HIV gp41, HIV gp120, HIV gp160, HIV P17/24, HIV P24, HIV P55 GAG, HIV P66 POL, HIV TAT, HIV GP36, the Nef protein and reverse transcriptase; an influenza antigen including, but not limited to, hemagglutinin and neuraminidase; a Japanese encephalitis viral antigen including, but not limited to, proteins E, M-E, M-E-NS 1, NS1, NS1-NS2A and 80% E; a measles antigen including, but not limited to, the measles virus fusion protein; a rabies antigen including, but not limited to, rabies glycoprotein and rabies nucleoprotein; a respiratory syncytial viral antigen including, but not limited to, the RSV fusion protein and the M2 protein; a rotaviral antigen including, but not limited to, VP7sc; a rubella antigen including, but not limited to, proteins E1 and E2; and a varicella zoster viral antigen including, but not limited to, gpl and gpll.

Additionally or alternatively, antigen or antigens associated with tumor antigen/s may be used to deplete alloreactive clones from the memory T cell pool, such antigens are discussed herein above (in relation to target cell antigens for tg-TCR and/or CAR).

According to one embodiment, the antigen comprises one antigen (e.g. viral, bacterial or tumor antigen).

According to one embodiment, the antigen or antigens comprise two or more antigens (e.g. a mixture of antigens of one group of antigens, e.g. viral antigens, tumor antigens, etc.; or a mixture of antigens from different groups of antigens, e.g. viral and bacterial antigens, viral and tumor antigens).

According to one embodiment, the antigen or antigens comprise one, two, three, four, five or more antigens (e.g. in a single formulation or in several formulations).

According to one embodiment, the antigen or antigens comprise one, two, three, four, five or more tumor antigens (e.g. in a single formulation or in several formulations).

According to one embodiment, the antigen or antigens comprise one, two, three, four, five or more viral antigens (e.g. in a single formulation or in several formulations).

According to one embodiment, the antigen or antigens comprise viral peptides.

According to a specific embodiment, the vial peptides comprise peptides (e.g. one or more peptides) from a single virus (i.e. from one virus type).

According to a specific embodiment, the vial peptides comprise peptides from two, three, four, five or more viruses (i.e. from different types of viruses).

According to a particular embodiment, the antigen or antigens comprise three viral antigens, e.g. an Epstein-Barr virus (EBV) peptide, a cytomegalovirus (CMV) peptide and an Adenovirus (Adv) peptide.

According to a particular embodiment, the antigen or antigens comprise four viral antigens, e.g. Epstein-Barr virus (EBV) peptide, a cytomegalovirus (CMV) peptide, a BK Virus peptide and an Adenovirus (Adv) peptide.

According to a specific embodiment, the viral peptides comprise at least one of EBV-LMP2, EBV-BZLF1, EBV-EBNA1, EBV-BRAF1, EBV-BMLF1, EBV-GP340/350 EBNA2, EBV-EBNA3a, EBV-EBNA3b, EBV-EBNA3c, CMV-pp65, CMV-IE-1, Adv-penton, Adv-hexon, BKV LT, BKV (capsid VP1), BKV (capsid protein VP2), BKV (capsid protein VP2, isoporm VP3), and BKV (small T antigen).

According to a specific embodiment, the viral peptides comprise two, three, four, five or more of EBV-LMP2, EBV-BZLF1, EBV-EBNA1, EBV-BRAF1, EBV-BMLF1, EBV-GP340/350 EBNA2, EBV-EBNA3a, EBV-EBNA3b, EBV-EBNA3c, CMV-pp65, CMV-IE-1, Adv-penton, Adv-hexon, BKV LT, BKV (capsid VP1), BKV (capsid protein VP2), BKV (capsid protein VP2, isoporm VP3), and BKV (small T antigen).

According to a particular embodiment, the antigen or antigens comprise a mixture of pepmixes which are overlapping peptide libraries (e.g. 15mers overlapping by 11 amino acids) spanning the entire protein sequence of three viruses: CMV, EBV, and Adeno (such pepmixes can be commercially bought e.g. from JPT Technologies, Berlin, Germany).

According to another particular embodiment, the antigen or antigens comprise a mixture of seven pepmixes spanning EBV-LMP2, EBV-BZLF1, EBV-EBNA1, CMV-pp65, CMV-IE-1, Adv-penton and Adv-hexon at a concentration of e.g. 100 ng/peptide or 700 ng/mixture of the seven peptides.

According to a particular embodiment, the antigen or antigens comprise viral peptides and bacterial peptides.

Additional viral and bacterial antigens which may be used to deplete alloreactive clones from the memory T cell pool are discussed herein above (in the section relating to target cell antigens for tg-TCR and/or CAR).

Dedicated software can be used to analyze viral, bacterial, fungal, tumor antigen sequences to identify immunogenic short peptides, e.g., peptides presentable in context of major histocompatibility complex (MHC) class I or MHC class II.

In order to stimulate an immune response of the memory CD8⁺ T cells, additional stimulatory antigens may be used such as, but not limited to, ovalbumin, DNP (dinitrophenyl), KLH (keyhole limpet hemocyanin).

According to one embodiment, the third party antigen or antigens comprise third party cells.

Third party cells can be either allogeneic or xenogeneic with respects to the donor and recipient (explained in further detail hereinbelow). In the case of allogeneic third party cells, such cells have HLA antigens different from that of the donor but which are not cross reactive with the recipient HLA antigens, such that veto cells generated against such cells are not reactive against a transplant or recipient antigens.

According to an embodiment of the present invention the allogeneic or xenogeneic third party cells are stimulatory cells selected from the group consisting of cells purified from peripheral blood lymphocytes (PBL), spleen or lymph nodes, cytokine-mobilized PBLs, in vitro expanded antigen-presenting cells (APC), in vitro expanded dendritic cells (DC) and artificial antigen presenting cells.

Antigens of the invention can be presented on the cellular, viral, fungal or bacterial surfaces or derived and/or purified therefrom. Additionally, a viral, fungal or bacterial antigen can be displayed on an infected cell or a cellular antigen can be displayed on an artificial vehicle (e.g. liposome, exosome) or on an artificial antigen presenting cell (e.g. cell line transfected with the antigen or antigens). Thus, viral, bacterial or fungal antigens can be presented by cells infected therewith or otherwise made to express viral/bacterial/fungi peptides. Similarly, tumor antigens can be presented by cells made to express these proteins.

Utilizing cells, virally infected cells, bacteria infected cells, viral peptides presenting cells or bacteria peptides presenting cells as antigens is particularly advantageous since such antigens include a diverse array of antigenic determinants and as such direct the formation of Tcm cells of a diverse population, which may further serve in faster reconstitution of T cells in cases where such reconstitution is required, e.g., following lethal or sublethal irradiation or chemotherapy procedure (as discussed in detail below) or to combat diseases (as discussed in detail below).

Thus, antigen presenting cells (autologous or non-autologous, as discussed below), cell lines, artificial vehicles (such as a liposome or exosome) or artificial antigen presenting cells (e.g. leukemic or fibroblast cell line transfected with the antigen or antigens), can be used to present short synthetic peptides fused or loaded thereto or to present protein extracts or purified proteins. Such short peptides, protein extracts or purified proteins may be viral-, bacterial-, fungal-, tumor-antigen derived peptides or peptides representing any other antigen.

According to one embodiment, the antigen or antigens are presented by antigen presenting cells (e.g. DCs) autologous with respect to the memory CD8⁺ T cells, e.g. of the same origin (e.g. of the same donor), in order to enable memory CD8⁺ T cell recognition in the context of MHC class I or MHC class II.

Additionally or alternatively, antigen or antigens (e.g. viral peptides) of some embodiments of the invention can be displayed on an artificial vehicle (e.g. liposome) or artificial APC. The artificial vehicles or artificial APC of the present invention may be engineered to exhibit MHC without being pulsed with an exogenous peptide. Thus, according to one embodiment, the artificial APC comprises K562 tumor cells transfected with a MHC determinant (e.g. autologous with respect to the memory CD8⁺ T cell) and a co-stimulatory molecule [as previously described e.g. Suhoski MM et al., Mol Ther. (2007) 15(5): 981-8], or fibroblasts transfected with same.

It will be appreciated that antigen presenting cells may express all of the antigens on a single cell or may express only part of the antigens on a single cell. Moreover, different antigen presenting cells (e.g. in the same preparation) may express different antigens. Accordingly, the antigen presenting cells (e.g. DC) comprise a heterogeneous cell mixture.

According to one embodiment, the antigen or antigens are presented by genetically modified antigen presenting cells or artificial antigen presenting cells exhibiting MHC antigens (also referred to as human leukocyte antigen (HLA)) recognizable by the memory CD8⁺ T cells.

According to one embodiment, the antigen presenting cells comprise human cells.

According to one embodiment, the antigen presenting cells comprise dendritic cells (DCs).

According to a specific embodiment, the antigen presenting cells comprise CD 14⁺-derived dendritic cells (DC).

According to a specific embodiment, the antigen presenting cells comprise mature dendritic cells (mDC).

According to a specific embodiment, the antigen presenting cells comprise irradiated dendritic cells.

Thus, according to one embodiment, the DCs are irradiated with about 5-10 Gy, about 10-20 Gy, about 20-30 Gy, about 20-40 Gy, about 20-50 Gy, about 10-50 Gy. According to a specific embodiment, the DCs are irradiated with about 10-50 Gy (e.g. 30 Gy).

Methods of utilizing dendritic cells as APCs are known in the art. Thus, as a non-limiting example, peripheral blood mononuclear cells (PBMCs) may be obtained from a cell donor [e.g. from the same cell donor as the memory CD8⁺ T cells]. Briefly, PBMCs are obtained from a cell donor and fractionated using Ficoll to obtain mononuclear cells (MNC). Stimulator cells are generated from the MNC fraction by selection of CD14⁺ expressing cells from the MNC fraction (e.g. using CD14-binding monoclonal antibodies), the CD14⁺ expressing cells are grown with dendritic cell maturation factors (e.g. comprising IL-4, GM-CSF, IFN-γ and LPS for 12-20 hours, e.g. for 16 hours). The mature dendritic cells (mDCs) are then loaded with antigen or antigens, e.g. viral peptides (e.g. EBV, CMV, BKV and Adenovirus), as discussed below, and are irradiated (e.g. by 25 Gy).

In order to present the antigen or antigens on APCs (e.g. mature DCs), the antigen or antigens (e.g. viral antigens) are co-cultured with the APCs (e.g. DCs) for about for 30 minutes to 3 hours (e.g. 1 hour) at 37° C. at 5% CO₂/O₂. For instance, DCs may be loaded with a cocktail of pepmixes (viral peptides) by incubation for about 1 hour at 37° C. at 5% CO₂/O₂. The antigen-loaded APCs (e.g. DCs) are then ready to use for generation of Tcm cells from the population of memory CD8⁺ T cells according to some embodiments of the invention.

It will be appreciated that culturing the population of cells enriched of memory CD8⁺ T cells with an antigen or antigens (e.g. viral antigens) under conditions which allow enrichment of tolerance-inducing cells (as discussed below), results in anti-disease activity, e.g. anti-viral activity, of the Tcm cells. Thus, according to one embodiment, the resultant Tcm cells are reactive against the antigen or antigens against which they were stimulated (i.e. antigen-specific cells).

According to some embodiments, culturing the population of cells comprising T cells with an antigen or antigens under conditions which allow enrichment of tolerance-inducing antigen-specific cells having a Tcm phenotype, is affected by a method comprising:
(a) contacting a population of cells comprising T cells with an antigen or antigens in the presence of IL-21 so as to allow enrichment of antigen reactive cells; and
(b) culturing the cells resulting from step (a) in the presence of IL-21, IL-15 and/or IL-7 so as to allow proliferation of cells comprising the Tcm phenotype.

Thus, the Tcm cells of the present invention are typically generated by first contacting a population of cells comprising T cells (e.g. enriched with memory CD8⁺ T cells) with an antigen or antigens (such as described above) in a culture supplemented with IL-21 (e.g. in an otherwise cytokine-free culture i.e., without the addition of any additional cytokines). This step is typically carried out for about 12-24 hours, about 12-36 hours, about 12-72 hours, 12-96 hours, 12-120 hours, about 24-36 hours, about 24-48 hours, about 24-72 hours, about 36-48 hours, about 36-72 hours, about 48-72 hours, about 48-96 hours, about 48-120 hours, 0.5-1 days, 0.5-2 days, 0.5-3 days, 0.5-5 days, 0.5-6 days, 0.5-7 days, 1-2 days, 1-3 days, 1-5 days, 1-6 days, 1-7 days, 1-10 days, 2-3 days, 2-4 days, 2-5 days, 2-6 days, 2-8 days, 3-4 days, 3-5 days, 3-7 days, 4-5 days, 4-8 days, 5-7 days, 6-8 days or 8-10 days or and allows enrichment of antigen reactive cells.

According to a specific embodiment, contacting a population of cells comprising T cells (e.g. enriched with memory CD8⁺T cells) with an antigen or antigens (such as described above) in a culture supplemented with IL-21 (otherwise cytokine-free culture) is affected for 1-5 days (e.g. 2-4 days, e.g. 3 days).

Contacting a population cells comprising T cells (e.g. enriched with of memory CD8⁺ T cells) with an antigen or antigens (such as described above) in a culture supplemented with IL-21 is typically carried out in the presence of about 0.001-3000 IU/ml, 0.01-3000 IU/ml, 0.1-3000 IU/ml, 1-3000 IU/ml, 10-3000 IU/ml, 100-3000 IU/ml, 1000-3000 IU/ml, 0.001-1000 IU/ml, 0.01-1000 IU/ml, 0.1-1000 IU/ml, 1-1000 IU/ml, 10-1000 IU/ml, 100-1000 IU/ml, 250-1000 IU/ml, 500-1000 IU/ml, 750-1000 IU/ml, 10-500 IU/ml, 50-500 IU/ml, 100-500 IU/ml, 250-500 IU/ml, 100-250 IU/ml, 0.1-100 IU/ml, 1-100 IU/ml, 10-100 IU/ml, 30-100 IU/ml, 50-100 IU/ml, 1-50 IU/ml, 10-50 IU/ml, 20-50 IU/ml, 30-50 IU/ml, 1-30 IU/ml, 10-30 IU/ml, 20-30 IU/ml, 10-20 IU/ml, 0.1-10 IU/ml, or 1-10 IU/ml IL-21. According to a specific embodiment, the concentration of IL-21 is 50-500 IU/ml (e.g. 100 IU/ml).

According to a specific embodiment, contacting a population of cells comprising T cells (e.g. enriched with memory CD8⁺ T cells) with an antigen or antigens is affected in a cytokine-free culture (e.g. supplemented with only IL-21), such a culture condition enables survival and enrichment of only those cells which undergo stimulation and activation by the antigen or antigens (i.e. of antigen reactive cells) as these cells secrete cytokines (e.g. IL-2) which enable their survival (other T cell clones are more prone to death by neglect under these culture conditions).

Accordingly, contacting a population of cells comprising T cells (e.g. enriched with memory CD8⁺T cells) with an antigen or antigens under these culture conditions enables depletion of GVH reactive cells.

The ratio of antigen or antigens (e.g. presented on APCs such as antigen pulsed dendritic cells) to memory T cells is typically about 1:2 to about 1:10 such as about 1:4, about 1:5, about 1:6, about 1:8 or about 1:10. According to a specific embodiment, the ratio of antigen or antigens (e.g. presented on APCs) to memory T cells is about 1:2 to about 1:8 (e.g. 1:5).

Next, the resultant memory T cells (i.e. after culture with IL-21) are cultured in the presence of IL-21, IL-15 and/or IL-7 in an antigen free environment (i.e. without the addition of supplementary antigen or antigens to the cell culture) so as to allow proliferation of cells comprising the Tcm phenotype. This step is typically carried out for about 12-24 hours, about 12-36 hours, about 12-72 hours, about 12-96 hours, about 12-120 hours, about 12-240 hours, 24-36 hours, 24-48 hours, about 24-72 hours, 24-96 hours, 24-120 hours, 24-240 hours, about 48-72 hours, about 48-120 hours, about 48-240 hours, about 96-240 hours, about 120-144 hours, about 120-240 hours, about 144-240 hours, about 0.5-1 days, about 0.5-2 days, about 0.5-3 days, about 0.5-5 days, about 0.5-10 days, about 0.5-20 days, about 1-2 days, about 1-3 days, about 1-4 days, about 1-6 days, about 1-8 days, about 1-10 days, about 1-15 days, about 2-3 days, about 2-4 days, about 2-5 days, about 2-6 days, about 2-8 days, about 2-10 days, about 4-5 days, about 4-6 days, about 4-8 days, about 4-10 days, about 4-12 days, about 4-20 days, about 5-6 days, about 5-7 days, about 5-8 days, about 5-10 days, about 5-15 days, about 6-7 days, about 6-8 days, about 6-10 days, about 7-8 days, about 7-9 days, about 7-10 days, about 7-13 days, about 7-15 days, about 8-10 days, about 10-12 days, about 10-14 days, about 12-14 days, about 14-16 days, about 14-18 days, about 16-18 days or about 18-20 days.

According to a specific embodiment, the resultant memory T cells (i.e. after culture with IL-21) are cultured in the presence of IL-21, IL-15 and IL-7 in an antigen free environment (i.e. without the addition of supplementary antigen or antigens to the cell culture) for about 12 hours to 20 days, e.g. about 4-20 days, e.g. about 4-12 days (e.g. 9 days).

This step is typically carried out in the presence of IL-21 at a concentration of about 0.001-3000 IU/ml, 0.01-3000 IU/ml, 0.1-3000 IU/ml, 1-3000 IU/ml, 10-3000 IU/ml, 100-3000 IU/ml, 1000-3000 IU/ml, 0.001-1000 IU/ml, 0.01-1000 IU/ml, 0.1-1000 IU/ml, 1-1000 IU/ml, 10-1000 IU/ml, 100-1000 IU/ml, 250-1000 IU/ml, 500-1000 IU/ml, 750-1000 IU/ml, 10-500 IU/ml, 50-500 IU/ml, 100-500 IU/ml, 250-500 IU/ml, 100-250 IU/ml, 0.1-100 IU/ml, 1-100 IU/ml, 10-100 IU/ml, 30-100 IU/ml, 50-100 IU/ml, 1-50 IU/ml, 10-50 IU/ml, 20-50 IU/ml, 30-50 IU/ml, 1-30 IU/ml, 10-30 IU/ml, 20-30 IU/ml, 10-20 IU/ml, 0.1-10 IU/ml, or 1-10 IU/ml IL-21. According to a specific embodiment, the concentration of IL-21 is 50-500 IU/ml (e.g. 100 IU/ml).

This step is typically further carried out in the presence of IL-15 at a concentration of about 0.001-3000 IU/ml, 0.01-3000 IU/ml, 0.1-3000 IU/ml, 1-3000 IU/ml, 10-3000 IU/ml, 100-3000 IU/ml, 125-3000 IU/ml, 1000-3000 IU/ml, 0.001-1000 IU/ml, 0.01-1000 IU/ml, 0.1-1000 IU/ml, 1-1000 IU/ml, 10-1000 IU/ml, 100-1000 IU/ml, 125-1000 IU/ml, 250-1000 IU/ml, 500-1000 IU/ml, 750-1000 IU/ml, 10-500 IU/ml, 50-500 IU/ml, 100-500 IU/ml, 125-500 IU/ml, 250-500 IU/ml, 250-500 IU/ml, 125-250 IU/ml, 100-250 IU/ml, 0.1-100 IU/ml, 1-100 IU/ml, 10-100 IU/ml, 30-100 IU/ml, 50-100 IU/ml, 1-50 IU/ml, 10-50 IU/ml, 20-50 IU/ml, 30-50 IU/ml, 1-30 IU/ml, 10-30 IU/ml, 20-30 IU/ml, 10-20 IU/ml, 0.1-10 IU/ml, or 1-10 IU/ml IL-15. According to a specific embodiment the concentration of IL-15 is 50-500 IU/ml (e.g. 125 IU/ml).

This step is typically further carried out in the presence of IL-7 at a concentration of about 0.001-3000 IU/ml, 0.01-3000 IU/ml, 0.1-3000 IU/ml, 1-3000 IU/ml, 10-3000 IU/ml, 30-3000 IU/ml, 100-3000 IU/ml, 1000-3000 IU/ml, 0.001-1000 IU/ml, 0.01-1000 IU/ml, 0.1-1000 IU/ml, 1-1000 IU/ml, 10-1000 IU/ml, 30-1000 IU/ml, 100-1000 IU/ml, 250-1000 IU/ml, 500-1000 IU/ml, 750-1000 IU/ml, 10-500 IU/ml, 30-500 IU/ml, 50-500 IU/ml, 100-500 IU/ml, 250-500 IU/ml, 100-250 IU/ml, 0.1-100 IU/ml, 1-100 IU/ml, 10-100 IU/ml, 30-100 IU/ml, 50-100 IU/ml, 1-50 IU/ml, 10-50 IU/ml, 20-50 IU/ml, 30-50 IU/ml, 1-30 IU/ml, 10-30 IU/ml, 20-30 IU/ml, 10-20 IU/ml, 0.1-10 IU/ml, or 1-10 IU/ml IL-7. According to a specific embodiment the concentration of IL-7 is 1-100 IU/ml (30 IU/ml).

It will be appreciated that residual antigen or antigens can be present in the cell culture after culture with IL-21 (i.e. in the Tcm proliferation step comprising, for example, the addition of IL-21, IL-15 and IL-7) and thus an antigen free environment relates to a cell culture without the addition of supplementary antigen or antigens.

According to one embodiment, the total length of culturing time for generating the Tcm cells is about 7, 8, 9, 10, 11, 12, 13, 14, 15, 16, 17, 18, 19 or 20 days (e.g. 12 days).

An additional step which may be carried out in accordance with the present teachings includes selection and removal of activated cells. Such a selection step aids in removal of potential host reactive T cells.

Isolating activated cells may be carried out in a two stage approach. In the first stage activated cells are selected before culturing the cells in the presence of IL-21, IL-15 and IL-7. This first stage is typically carried out after the initial contacting of the memory T cells with an antigen or antigens in the presence of IL-21. This selection process picks only those cells which were activated by antigen or antigens (e.g. express activation markers as described below) and is typically affected about 12-24 hours, about 24-36 hours, about 12-36 hours, about 36-48 hours, about 12-48 hours, about 48-60 hours, about 12-60 hours, about 60-72 hours, about 12-72 hours, about 72-84 hours, about 12-84 hours, about 84-96 hours, about 12-96 hours, after the initial contacting of the memory T cells with an antigen or antigens. According to a specific embodiment, the selection process is affected about 12-24 hours (e.g. 14 hours) after the initial contacting of the memory T cells with an antigen or antigens.

Isolating activated cells may be affected by affinity based purification (e.g. such as by the use of MACS beads, FACS sorter and/or capture ELISA labeling) and may be affected towards any activation markers including cell surface markers such as, but not limited to, CD69, CD44, CD25, CFSE, CD137 or non-cell surface markers such as, but not limited to, IFN-γ and IL-2. Isolating activated cells may also be affected by morphology based purification (e.g. isolating large cells) using any method known in the art (e.g. by FACS). Typically, the activated cells are also selected for expression of CD8⁺ cells. Furthermore, any combination of the above methods may be utilized to efficiently isolate activated cells.

According to an embodiment of the present invention, selecting for activated cells is affected by selection of CD137+ and/or CD25+ cells.

The second stage of isolation of activated cells is typically carried out at the end of culturing (i.e. after culturing in an antigen free environment with IL-21, IL-15 and IL-7). This stage depletes alloreactive cells by depletion of those cells which were activated following contacting of the central memory T-lymphocyte (Tcm) with irradiated host antigen presenting cells (APCs e.g. dendritic cells). As mentioned above, isolating activated cells may be affected by affinity based purification (e.g. such as by the use of MACS beads, FACS sorter and/or capture ELISA labeling) and may be affected towards any activation markers including cell surface markers such as, but not limited to, CD69, CD44, CD25, CFSE, CD137 or non-cell surface markers such as, but not limited to, IFN-γ and IL-2.

According to an embodiment of the present invention, depleting the alloreactive cells is affected by depletion of CD137+ and/or CD25+ cells.

According to an embodiment of the present invention, depleting the alloreactive cells is affected by culturing the Tcm cells with irradiated host antigen presenting cells (APCs e.g. dendritic cells) for e.g. 12-24 hours (e.g. 16 hours) about 6-9 days (e.g. on day 8) from the beginning of culture (i.e. day 0 being the first day of culturing the memory CD8⁺ T cells with an antigen or antigens).

According to one embodiment, isolation of activated cells is carried out only by use of the first stage as discussed above.

According to another embodiment, isolation of activated cells is carried out only by use of the second stage as discussed above.

According to one embodiment, the veto cells comprising the Tcm phenotype comprise a CD3⁺, CD8⁺, CD62L⁺, CD45RA⁻, CD45RO⁺ signature.

It will be appreciated that at least 30%, at least 40%, at least 50%, at least 55%, at least 60%, at least 65%, at least 70%, at least 75%, at least 80%, at least 85%, at least 90%, at least 95% or even 100% of the veto cells are CD3⁺CD8⁺ cells. According to a specific embodiment, the veto cells comprise about 30-50% CD3⁺CD8⁺ cells. According to a specific embodiment, the veto cells comprise about 50-70% CD3⁺CD8⁺ cells. According to a specific embodiment, the veto cells comprise about 70-90% CD3⁺CD8⁺ cells.

It will be appreciated that at least 30%, at least 40%, at least 50%, at least 55%, at least 60%, at least 65%, at least 70%, at least 75%, at least 80%, at least 85%, at least 90%, at least 95% or even 100% of the CD3⁺CD8⁺ cells have the Tcm cell signature. According to a specific embodiment, about 30-80% of the CD3⁺CD8⁺ cells have the Tcm cell signature (e.g. 40-50%).

According to one embodiment, at least 50% of the cells are CD3⁺CD8⁺ cells of which at least 30% have the signature.

According to one embodiment, at least 50% of the cells are CD3⁺CD8⁺ cells of which at least 50% have the signature.

According to one embodiment, at least 50% of the cells are CD3⁺CD8⁺ cells of which at least 70% have the signature.

According to one embodiment, the non-GvHD inducing veto cells having a central memory T-lymphocyte (Tcm) phenotype of the invention are not naturally occurring and are not a product of nature. These cells are typically produced by ex-vivo manipulation (i.e. exposure to an antigen or antigens in the presence of specific cytokines).

According to one embodiment of the invention, there is provided a method of generating a population of non-GvHD inducing veto cells comprising a central memory T-lymphocyte (Tcm) phenotype, the cells being tolerance-inducing cells and/or endowed with anti-disease activity, and capable of homing to the lymph nodes following transplantation, the method comprising:
(a) providing a population of cells comprising T cells, the T cells comprising at least 40% CD8⁺ memory T cells;
(b) contacting the population of cells comprising T cells with an antigen or antigens in the presence of IL-21 so as to allow enrichment of antigen reactive cells (and depletion of GVH reactive cells); and
(c) culturing the cells resulting from step (b) in the presence of IL-21, IL-15 and/or IL-7 so as to allow proliferation of cells comprising the Tcm phenotype.

According to one embodiment of the invention, there is provided a method of generating a population of non-GvHD inducing veto cells comprising a central memory T-lymphocyte (Tcm) phenotype, the cells being tolerance-inducing cells and/or endowed with anti-disease activity, and capable of homing to the lymph nodes following transplantation, the method comprising:
(a) treating peripheral blood mononuclear cells (PBMCs) with an agent capable of depleting CD4⁺, CD56⁺ and CD45RA⁺ cells, or with an agent capable of selecting CD45RO⁺, CD8⁺ cells, so as to obtain a population of cells comprising T cells enriched of memory CD8⁺ T cells comprising a CD45RO⁺CD45RA⁻CD8⁺ phenotype;
(b) contacting the population of cells comprising T cells with the antigen or antigens in the presence of IL-21 so as to allow enrichment of antigen reactive cells; and
(c) culturing the cells resulting from step (b) in the presence of IL-21, IL-15 and/or IL-7 so as to allow proliferation of cells comprising the Tcm phenotype.

According to one embodiment of the invention, there is provided a method of generating a population of non-GvHD inducing veto cells comprising a central memory T-lymphocyte (Tcm) phenotype, the cells being tolerance-inducing cells and/or endowed with anti-viral activity, and capable of homing to the lymph nodes following transplantation, the method comprising:
(a) providing a population of cells comprising T cells, the T cells comprising at least 40% CD8⁺ memory T cells;
(b) contacting the population of cells comprising T cells with viral antigen or antigens in the presence of IL-21 so as to allow enrichment of viral antigen reactive cells; and
(c) culturing the cells resulting from step (b) in the presence of IL-21, IL-15 and/or IL-7 so as to allow proliferation of cells comprising the Tcm phenotype.

According to one embodiment of the invention, there is provided a method of generating a population of non-GvHD inducing veto cells comprising a central memory T-lymphocyte (Tcm) phenotype, the cells being tolerance-inducing cells and/or endowed with anti-viral activity, and capable of homing to the lymph nodes following transplantation, the method comprising:
(a) treating peripheral blood mononuclear cells (PBMCs) with an agent capable of depleting CD4⁺, CD56⁺ and CD45RA⁺ cells, or with an agent capable of selecting CD45RO⁺, CD8⁺ cells, so as to obtain a population of cells comprising T cells enriched of memory CD8⁺ T cells comprising a CD45RO⁺CD45RA⁻CD8⁺ phenotype;
(b) contacting the population of cells comprising T cells with viral antigen or antigens in the presence of IL-21 so as to allow enrichment of viral antigen reactive cells; and
(c) culturing the cells resulting from step (b) in the presence of IL-21, IL-15 and/or IL-7 so as to allow proliferation of cells comprising the Tcm phenotype.

As mentioned above, contacting a population of cells with an antigen or antigens (e.g. viral antigens) in the presence of IL-21 allows enrichment of antigen (e.g. viral reactive cells) while concomitantly enabling depletion of GVH reactive cells.

According to one embodiment, in order to obtain memory T cells specific to an antigen or antigens, the antigen/s (e.g. tumor antigen, viral antigen) is administered to the memory T cell donor prior to obtaining memory T cells therefrom (e.g. prior to providing the population of cells comprising T cells, the T cells comprising at least 40% memory T cells). Any method of immunizing a cell donor against an antigen in order to elicit an immunogenic response (e.g. generation of memory T cells) may be employed.

The antigen may be administered as is or as part of a composition comprising an adjuvant (e.g. Complete Freund’s adjuvant (CFA) or Incomplete Freund’s adjuvant (IFA)). According to one embodiment, the antigen is administered to a memory T cell donor once. According to one embodiment, the memory T cell donor receives at least one additional (e.g. boost) administration of the antigen (e.g. 2, 3, 4 or more administrations). Such an additional administration may be affected 1, 3, 5, 7, 10, 12, 14, 21, 30 days or more following the first administration of the antigen.

Additional methods of immunizing a subject towards a tumor antigen which can be used with some embodiments of the invention (e.g. cell based vaccines such as peptide-specific DC vaccines, DC vaccines against undefined epitopes, using leukemia-derived DCs for vaccination, GVAX^{®} platform) are described in Alatrash G. and Molldrem J., Expert Rev Hematol. (2011) 4(1): 37-50, incorporated herein by reference.

In order to further enrich the memory T cells against a particular antigen/s and to deplete alloreactive clones from the memory T cell pool, the memory T cells may be further contacted with the same antigen or antigens (e.g. the same antigen as administered to the cell donor), as described hereinabove.

As mentioned, the veto Tcm cells of the invention are transduced with a polynucleotide encoding a cell surface receptor comprising a T cell receptor signaling module.

According to one embodiment, transducing is affected concomitantly with culturing of the population of cells comprising T cells with an antigen or antigens under conditions which allow enrichment of tolerance-inducing antigen-specific cells having a Tcm phenotype.

According to one embodiment, transducing is typically affected on days 3-9 of culture (e.g. on days 3-8 of culture, e.g. on days 3-7 of culture, e.g. on days 4-8 of culture, e.g. on days 4-7 of culture, e.g. on days 4-6 of culture, e.g. on days 5-7 of culture, e.g. on days 5-6 of culture). According to a specific embodiment, transducing is typically affected on days 5-6 of culture.

Accordingly, the Tcm cells are typically transduced with a polynucleotide encoding a cell surface receptor comprising a T cell receptor signaling module at the stage when the cells are cultured in the presence of IL-21, IL-15 and/or IL-7, i.e. in the Tcm proliferation stage of culture.

According to one embodiment, transducing is affected prior to culturing of the population of cells comprising T cells with an antigen or antigens under conditions which allow enrichment of tolerance-inducing antigen-specific cells having a Tcm phenotype.

According to one embodiment, transducing the population of cells comprising T cells is effected on fresh cells.

According to one embodiment, transducing the population of cells comprising T cells is effected on CD4⁻CD56⁻CD45RA⁻ cells.

Accordingly, the population of cells comprising T cells (e.g. comprising at least 40% memory CD8⁺ cells) may be transduced with a polynucleotide encoding a cell surface receptor comprising a T cell receptor signaling module prior to culturing of the cells with an antigen or antigens under conditions which allow enrichment of tolerance-inducing antigen specific cells having a Tcm phenotype and being depleted of GVH reactivity (i.e. veto cells). Under such conditions, transducing is typically affected within 1-7 days, e.g. 3-4 days, of obtaining the cells (e.g. of leukapheresis).

According to one embodiment the population of cells comprising T cells are transduced on days 1-3 of culture (e.g. on days 1, 2 or 3 of culture) and thereafter the resultant population of cells are cultured with the antigen or antigens in the presence of IL-21 (e.g. for about 2-4 days, e.g. for 3 days) followed by culture with IL-21, IL-7 and IL-15 (e.g. for about 4-12 days, e.g. 9 days).

According to one embodiment, transducing the population of cells comprising T cells with a polynucleotide encoding a cell surface receptor comprising a T cell receptor signaling module is affected using methods not requiring activation of the cells to enter the cell cycle (e.g. proliferation), e.g. utilizing a lentivirus or by gene editing as discussed below.

According to one embodiment, transducing is affected following culturing of the population of cells comprising T cells with an antigen or antigens under conditions which allow enrichment of tolerance-inducing antigen-specific cells having a Tcm phenotype and being depleted of GVH reactivity (i.e. veto cells).

Accordingly, the veto cells may be transduced with a polynucleotide encoding a cell surface receptor comprising a T cell receptor signaling module. Under such conditions, transducing may be affected at any time after generation of the veto cells, e.g. within 12, 24, 36, 48 hours, or within 1, 2, 3, 4, 5, 6, 7, 10, 12, 14, 21, 30, 45, 60, 90 days or more after generation of the veto cells.

According to one embodiment, transducing the veto cells with a polynucleotide encoding a cell surface receptor comprising a T cell receptor signaling module is affected using methods not requiring activation of the cells to enter the cell cycle (e.g. proliferation), e.g. utilizing a lentivirus or by gene editing as discussed below.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

The isolated polynucleotide can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The polynucleotide according to some embodiments of the invention may comprise a single polynucleotide comprising a nucleic acid sequence encoding the extracellular domain, the transmembrane domain and/or the signaling module of the cell surface receptor (e.g. tg-TCR and/or CAR). Alternatively, two or more polynucleotides may be used wherein one polynucleotide may comprise a nucleic acid sequence which encodes, for example, the extracellular domain and transmembrane domain and another polynucleotide may comprise a nucleic acid sequence which encodes the signaling module.

According to an aspect of some embodiments of the invention there is provided a nucleic acid construct comprising an isolated polynucleotide comprising a nucleic acid sequence encoding the molecule of some embodiments of the invention and a cis-acting regulatory element for directing transcription of the isolated polynucleotide in a host cell.

Thus, the expression of natural or synthetic nucleic acids encoding the cell surface receptor (e.g. tg-TCR or CAR molecule) of the invention is typically achieved by operably linking a nucleic acid encoding the cell surface receptor (e.g. tg-TCR or CAR) polypeptide or portions thereof to a cis-acting regulatory element (e.g., a promoter sequence), and incorporating the construct into an expression vector.

The nucleic acid construct of the invention may also include an enhancer, a transcription and translation initiation sequence, transcription and translation terminator and a polyadenylation signal, a 5′ LTR, a tRNA binding site, a packaging signal, an origin of second-strand DNA synthesis, and a 3′ LTR or a portion thereof; additional polynucleotide sequences that allow, for example, the translation of several proteins from a single mRNA such as an internal ribosome entry site (IRES) and sequences for genomic integration of the promoter-chimeric polypeptide; sequences engineered to enhance stability, production, purification, yield or toxicity of the expressed peptide.

Enhancers regulate the frequency of transcriptional initiation. Typically, promoter elements are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1.alpha. (EF-1.alpha.). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionein promoter, a glucocorticoid promoter, a progesterone promoter, and a tetracycline promoter.

The isolated polynucleotide of the invention can be cloned into a number of types of vectors. For example, the isolated polynucleotide can be cloned into a vector including, but not limited to a plasmid, a phagemid, a phage derivative, an animal virus, and a cosmid. Vectors of particular interest include expression vectors, replication vectors, probe generation vectors, and sequencing vectors.

Examples for mammalian expression vectors include, but are not limited to, pcDNA3, pcDNA3.1(+/-), pGL3, pZeoSV2(+/-), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p205. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Current in vivo or in vitro nucleic acid transfer techniques include transfection with viral or non-viral constructs, such as adenovirus, lentivirus, Herpes simplex I virus, or adeno-associated virus (AAV). Recombinant viral vectors offer advantages such as lateral infection and targeting specificity. Introduction of nucleic acids by viral infection offers several advantages over other methods such as lipofection and electroporation, since higher transfection efficiency can be obtained due to the infectious nature of viruses.

Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

According to some embodiments of the invention, the nucleic acid construct of the invention is a viral vector.

Vectors derived from retroviruses such as the lentivirus are suitable tools to achieve long-term gene transfer since they allow long-term, stable integration of a transgene and its propagation in daughter cells. Lentiviral vectors have the added advantage over vectors derived from oncoretroviruses such as murine leukemia viruses in that they can transduce non-proliferating cells, such as hepatocytes.

Furthermore, lentiviral vectors offer a larger gene insertion capacity and also have the added advantage of low immunogenicity. Alternatively, gamma-retroviral vectors may be used. Gamma-retroviral vectors have good transduction efficiency and no vector-associated toxicity [see e.g. Zhang and Morgan, Adv Drug Deliv Rev. (2012) supra].

For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo.

In order to assess the expression of a cell surface receptor (e.g. tg-TCR or CAR) polypeptide or portions thereof, the nucleic acid construct to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tel et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Various methods can be used to introduce the nucleic acid construct of the invention into a host cell, e.g., mammalian, bacterial, yeast, or insect cell. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, physical, chemical, or biological means (e.g., stable or transient transfection, lipofection, electroporation and infection with recombinant viral vectors). In addition, see U.S. Pat. Nos. 5,464,764 and 5,487,992 for positive-negative selection methods.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Biological methods for introducing a polynucleotide of interest into a host cell include the use of DNA and RNA vectors (as described above). Viral vectors, and especially retroviral vectors, have become the most widely used method for inserting genes into mammalian, e.g., human cells. Other viral vectors can be derived from lentivirus, poxviruses, herpes simplex virus 1, adenoviruses and adeno-associated viruses, and the like. See, for example, U.S. Pat. Nos. 5,350,674 and 5,585,362.

In the case where a non-viral delivery system is utilized, an exemplary delivery vehicle is a liposome.

“Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution.

The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as non-uniform aggregates of lipid molecules. Also contemplated are lipofectamine-nucleic acid complexes.

The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”); and other lipids may be obtained from Avanti Polar Lipids, Inc, (Birmingham, Ala.). Additionally or alternatively, the DOTMA, DOPE, and DC-Chol [Tonkinson et al., Cancer Investigation, 14(1): 54-65 (1996)] lipids can be used. Stock solutions of lipids in chloroform or chloroform/methanol can be stored at about -20.degree. C. Chloroform is used as the only solvent since it is more readily evaporated than methanol.

Another exemplary non-viral delivery system which may be used in accordance with the present invention is a transposon-based non-viral gene delivery system, such as e.g. Sleeping Beauty or PiggyBac.

Another exemplary non-viral delivery system which may be used in accordance with the present invention is a gene editing system such as CRISPR/Cas system. This system comprises the clustered regularly interspaced short palindromic repeat (CRISPR) genes that produce RNA components and CRISPR associated (Cas) genes that encode protein components. Moreover, the CRIPSR/Cas system for genome editing typically comprises two distinct components: a gRNA and an endonuclease e.g. Cas9.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

It will be appreciated that the cell transduced with the cell surface receptor (e.g. tg-TCR and/or CAR) may further be genetically modified to repress expression of at least one endogenous immunological checkpoint gene in the cell.

The immunological checkpoint gene may comprise a PD or CTLA gene.

As used herein the term “immunological checkpoint gene” refers to any gene that is involved in an inhibitory process (e.g., feedback loop) that acts to regulate the amplitude of an immune response, for example an immune inhibitory feedback loop that mitigates uncontrolled propagation of harmful immune responses.

Non-limiting examples of immunological checkpoint genes include members of the extended CD28 family of receptors and their ligands as well as genes involved in co-inhibitory pathways (e.g., CTLA-4 and PD-1).

Thus, according to one embodiment PD1 and/or CTLA-4-targeted nucleases or transcription repressors can be utilized as discussed in U.S. Pat. Application No. 20140120622, incorporated herein by reference.

Additionally or alternatively, immune checkpoint proteins, which regulate activation or function of a T cell, including for example, PD1, PDL-1, B7H2, B7H4, CTLA-4, CD80, CD86, LAG-3, TIM-3, KIR, IDO, CD19, OX40, 4-1BB (CD137), CD27, CD70, CD40, GITR, CD28 and/or ICOS (CD278), may be modulated (e.g. upregulated or downregulated as needed) in the transduced cell by the use of an immune checkpoint regulator.

According to specific embodiments, the immune-check point regulator is selected from the group consisting of anti-CTLA4, anti-PD-1, anti-PDL-1, CD40 agonist, 4-1BB agonist, GITR agonist and OX40 agonist.

According to an aspect of some embodiments of the invention there is provided an isolated population of genetically modified veto cells obtainable according to the methods of some embodiments of the invention.

According to one embodiment, the isolated population of genetically modified veto cells comprise at least 50%, 60%, 70%, 75%, 80%, 85%, 90% or 95% or more CD8⁺ T cells of which at least 50%, 60%, 70%, 75%, 80%, 85%, 90% or 95% or more express the heterologous cell surface receptor comprising a T cell receptor signaling module (e.g. CAR or tg-TCR).

According to a specific embodiment, the isolated population of genetically modified veto cells comprise at least about 30%, 40%, 50%, 60%, 70%, 80% CD8⁺ T cells (e.g. 50% CD8⁺ T cells, e.g. 70% CD8⁺ T cells) of which at least about 10%, 20%, 30%, 40%, 50%, or more cells (e.g. 50%) express the heterologous cell surface receptor comprising a T cell receptor signaling module (CAR or tg-TCR).

According to one embodiment, the isolated population of genetically modified veto cells are tolerance-inducing cells, exhibit a specific reactivity against their target antigen (e.g. tumor antigen) via the transduced CAR or tg-TCR, and comprise anti-disease activity (e.g. anti-viral activity) by virtue of their culturing against third party antigen/s (e.g. viral antigen/s) used for generation of Tcm cells and for elimination of anti-host clones. It will be appreciated that the veto CAR-T cells of some embodiments of the invention utilize both TCR-dependent and TCR-independent mechanisms in order to exert their therapeutic effect.

The isolated population of genetically modified veto cells of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the genetically modified veto Tcm cells accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington’s Pharmaceutical Sciences”, Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method. Alternately, one may administer the pharmaceutical composition in a local rather than systemic manner, for example, via injection of the pharmaceutical composition directly into a tissue region of a patient.

According to one embodiment, the route of administration includes, for example, an injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the pharmaceutical composition of the present invention is administered to a patient by intradermal or subcutaneous injection. In another embodiment, the pharmaceutical composition of the present invention is preferably administered by i.v. injection. The pharmaceutical composition may be injected directly into a tumor, lymph node, or site of infection.

Pharmaceutical compositions of some embodiments of the invention may be manufactured by processes well known in the art, e.g., by means of conventional mixing, dissolving, granulating, dragee-making, levitating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank’s solution, Ringer’s solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion.

Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes.

Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (genetically modified veto Tcm cells) effective to prevent, alleviate or ameliorate symptoms of a disorder (e.g., malignant or non-malignant disease) or prolong the survival of the subject being treated.

Determination of a therapeutically effective amount is well within the capability of those skilled in the art, especially in light of the detailed disclosure provided herein.

When “therapeutic amount” is indicated, the precise amount of the compositions of the present invention to be administered can be determined by a physician with consideration of individual differences in age, weight, disease state, e.g. tumor size, extent of infection or metastasis, and the condition of the patient (subject). It can generally be stated that a pharmaceutical composition comprising the cells described herein may be administered at a dosage of 10⁴ to 10⁹ cells/kg body weight, including all integer values within those ranges.

For example, the number of cells infused to a recipient should be more than 1 x 10⁴ /Kg body weight. The number of cells infused to a recipient should typically be in the range of 1 x 10³ /Kg body weight to 1 x 10⁴ /Kg body weight, range of 1 x 10⁴ /Kg body weight to 1 x 10⁵ /Kg body weight, range of 1 x 10⁴ /Kg body weight to 1 x 10⁶ /Kg body weight, range of 1 x 10⁴ /Kg body weight to 10 x 10⁷ /Kg body weight, range of 1 x 10⁴ /Kg body weight to 1 x 10⁸ /Kg body weight, range of 1 x 10³ /Kg body weight to 1 x 10⁵ /Kg body weight, range of 1 x 10⁴ /Kg body weight to 1 x 10⁶ /Kg body weight, range of 1 x 10⁶ /Kg body weight to 10 x 10⁷ /Kg body weight, range of 1 x 10⁵ /Kg body weight to 10 x 10⁷ /Kg body weight, range of 1 x 10⁶ /Kg body weight to 1 x 10⁸ /Kg body weight, or range of 1 x 10⁶ /Kg body weight to 1 x 10⁹ /Kg body weight. According to a specific embodiment, the number of cells infused to a recipient should be in the range of 1 x 10⁶ /Kg body weight to 10 x 10⁸ /Kg body weight.

The cell compositions of some embodiments of the invention may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

For example, the effect of the active ingredients (e.g., the genetically modified veto Tcm cells of some embodiments of the invention) on the pathology can be evaluated by monitoring the level of cellular markers, hormones, glucose, peptides, carbohydrates, cytokines, etc. in a biological sample of the treated subject using well known methods (e.g. ELISA, FACS, etc.) or by monitoring the tumor size using well known methods (e.g. ultrasound, CT, MRI, etc).

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human.

The dosage may vary depending upon the dosage form employed and the route of administration utilized. The exact formulation, route of administration and dosage can be chosen by the individual physician in view of the patient’s condition. (See e.g., Fingl, et al., 1975, in “The Pharmacological Basis of Therapeutics”, Ch. 1 p.1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

According to some embodiments of the invention, the therapeutic agent of the invention can be provided to the subject in conjunction with other drug(s) designed for treating the pathology [i.e. combination therapy, e.g., before, concomitantly with, or following administration of the genetically modified veto cells].

In certain embodiments of the present invention, the cells of some embodiments of the invention are administered to a patient in conjunction with any number of relevant treatment modalities, including but not limited to treatment with agents such as antiviral agents (e.g. Ganciclovir, Valaciclovir, Acyclovir, Valganciclovir, Foscarnet, Cidofovir, Maribavir, Leflunomide); chemotherapeutic agents (e.g. antineoplastic agents, such as but not limited to, Alkylating agents including e.g. Cyclophosphamide, Busulfan, Mechlorethamine or mustine (HN2), Uramustine or uracil mustard, Melphalan, Chlorambucil, Ifosfamide, Bendamustine, Nitrosoureas Carmustine, Lomustine, Streptozocin, Thiotepa, Cisplatin, Carboplatin, Nedaplatin, Oxaliplatin, Satraplatin, Triplatin tetranitrate, Procarbazine, Altretamine, Triazenes (dacarbazine, mitozolomide, temozolomide), Dacarbazine, Temozolomide, Myleran, Busulfex, Fludarabine, Dimethyl mileran or Cytarabine); agents for the treatment of MS (e.g. natalizumab); or agents for the treatment of psoriasis (e.g. efalizumab).

In further embodiments, the genetically modified veto Tcm cells of some embodiments of the invention may be used in combination with chemotherapy, radiation therapy, immunosuppressive agents (e.g. cyclosporin, azathioprine, methotrexate, mycophenolate, and FK506), antibodies, or other immunoablative agents (discussed in further detail below).

In a further embodiment, the genetically modified veto Tcm cell compositions of some embodiments of the invention are administered to a patient in conjunction with (e.g., before, concomitantly with, or following) immature hematopoietic cell (e.g. bone marrow) transplantation.

In a further embodiment, the genetically modified veto Tcm cell compositions of some embodiments of the invention are administered to a patient in conjunction with (e.g., before, concomitantly with, or following) non-transduced veto cells (i.e. not transduced to express a heterologous cell surface receptor comprising a T cell receptor signaling module, e.g. CAR or tg-TCR).

In a further embodiment, the genetically modified veto Tcm cell compositions of some embodiments of the invention are administered to a patient following a T cell ablative therapy (also referred to as T cell debulking) using, for example, chemotherapy agents such as, fludarabine, external-beam radiation therapy (XRT), cyclophosphamide, or antibodies such as anti-thymocyte globulin (ATG) antibodies, anti-CD3 (OKT3) antibodies or CAMPATH^{®} (Alemtuzumab, anti-CD52 antibodies).

In another embodiment, the genetically modified veto Tcm cell compositions of the present invention are administered following B-cell ablative therapy such as agents that react with CD20, e.g., Rituxan.

The combination therapy may increase the therapeutic effect of the agent of the invention in the treated subject.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The kit may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to one embodiment, the kit further comprises a chemotherapeutic agent (e.g. antineoplastic agent, as discussed in detail hereinbelow).

According to one embodiment, the kit further comprises an antiviral agent (as discussed in detail herein above).

According to an aspect of some embodiments of the invention, there is provided a method of treating a disease in a subject in need thereof, the method comprising administering to the subject a therapeutically effective amount of the isolated population genetically modified veto Tcm cells of some embodiments of the invention, thereby treating the subject.

According to an aspect of some embodiments of the invention, there is provided a therapeutically effective amount of the isolated population genetically modified veto Tcm cells of some embodiments of the invention for use in treating a disease in a subject in need thereof.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “subject” or “subject in need thereof” refers to a mammal, preferably a human being, male or female at any age that is in need of a cell or tissue transplantation or suffers from a disease which may be treated with the genetically modified veto cells.

According to one embodiment, the subject is in need of cell or tissue transplantation (also referred to herein as recipient) due to a disorder or a pathological or undesired condition, state, or syndrome, or a physical, morphological or physiological abnormality which is amenable to treatment via cell or tissue transplantation. Examples of such disorders are provided further below.

Thus, the method of the present invention may be applied to treat any disease such as, but not limited to, a malignant disease (e.g. cancer), a disease associated with transplantation of a graft (e.g. graft rejection, graft versus host disease), an infectious disease (e.g. viral infection, bacterial infection, fungal infection, protozoan infection or parasitic infections), a non-malignant hematologic disease.

According to one embodiment, the subject has a malignant disease.

### Cancerous Diseases

Malignant diseases (also termed cancers) which can be treated by the method of some embodiments of the invention can be any solid or non-solid tumor and/or tumor metastasis.

Examples of cancer include, but are not limited to, carcinoma, lymphoma, blastoma, sarcoma, and leukemia. More particular examples of such cancers include squamous cell cancer, soft-tissue sarcoma, Kaposi’s sarcoma, melanoma, lung cancer (including small-cell lung cancer, non-small-cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, rectal cancer, endometrial or uterine carcinoma, carcinoid carcinoma, salivary gland carcinoma, kidney or renal cancer, liver cancer, prostate cancer, vulvar cancer, thyroid cancer, hepatic carcinoma, mesothelioma, multiple myeloma, post-transplant lymphoproliferative disorder (PTLD), and various types of head and neck cancer (e.g. brain tumor). The cancerous conditions amenable for treatment of the invention include metastatic cancers.

According to one embodiment, the malignant disease is a hematological malignancy. Exemplary hematological malignancies include, but are not limited to, leukemia [e.g., acute lymphatic, acute lymphoblastic, acute lymphoblastic pre-B cell, acute lymphoblastic T cell leukemia, acute - megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia, T-cell acute lymphocytic leukemia (T-ALL) and B-cell chronic lymphocytic leukemia (B-CLL)] and lymphoma [e.g., Hodgkin’s disease, non-Hodgkin’s lymphoma, Burkitt, cutaneous T cell, histiocytic, lymphoblastic, T cell, thymic, B cell, including low grade/follicular; small lymphocytic (SL) NHL; intermediate grade/follicular NHL; intermediate grade diffuse NHL; high grade immunoblastic NHL; high grade lymphoblastic NHL; high-grade small non-cleaved cell NHL; bulky disease NHL; mantle cell lymphoma; AIDS-related lymphoma; and Waldenstrom’s Macroglobulinemia].

According to a specific embodiment, the malignant disease is a leukemia, a lymphoma, a myeloma, a melanoma, a sarcoma, a neuroblastoma, a colon cancer, a colorectal cancer, a breast cancer, an ovarian cancer, an esophageal cancer, a synovial cell cancer or a pancreatic cancer.

According to some embodiments of the invention, the pathology is a solid tumor.

According to some embodiments of the invention, the pathology is a tumor metastasis.

According to some embodiments of the invention, the pathology is a hematological malignancy.

According to a specific embodiment, the malignant disease is leukemia or a lymphoma.

According to a specific embodiment, the malignant disease is a multiple myeloma.

Exemplary malignant diseases which are treatable by the methods of some embodiments of the invention are listed in Tables 2 and 3, below.

TABLE 2

Clinical applications utilizing tg-TCR transduced veto cells with optional preconditioning regimens

| Target Ag of tg-TCR | Disease | Preconditioning |
|---|---|---|
| MART-1 | melanoma | Cy + Flud (cyclophosphamide + fludarabine) |
| MART-1 | melanoma | Cy + Flud |
| gp100 | melanoma | Cy + Flud |
| p53/gp100 | breast cancer melanoma esophageal cancer | Cy + Flud |
| CEA | colorectal cancer | Cy + Flud |
| NY-ESO-1 | melanoma synovial cell cancer | Cy + Flud |
| MAGE-A3 | melanoma synovial cell cancer esophageal cancer | Cy + Flud |
| MAGE-A3 | melanoma myeloma | Cy |
| | | Melphalan and auto stem cell transplantation (SCT) |

(adapted from Fujiwara, Pharmaceuticals (2014), 7: 1049 -1068)

TABLE 3

Clinical applications utilizing CAR transduced veto cells with optional preconditioning regimens

| | | |
|---|---|---|
| Target Ag of CAR | Disease | Preconditioning |
| L1-cell adhesion molecule | neuroblastoma | none |
| HER2 | colon cancer with lung/liver metastasis | Cy + Flud |
| GD2 | neuroblastoma | none |
| CD19 | Chronic lymphocytic leukemia (CLL) | CTx for CLL |
| CD19 | CLL Acute lymphocytic leukemia (ALL) | none Cy (1500 mg or 3000 mg) |
| CD19 | CLL follicular cell lymphoma (FL) | Cy + Flud |
| CD19 | B-cell acute lymphoblastic leukemia (B-ALL) | Cy (1500 mg or 3000 mg) |
| CD19 | ALL | CTx for ALL |
| CD19 | refractory B-ALL ph+ | Cy (1500 mg or 3000 mg) |
| CD20 | Mantle cell lymphoma (MCL) FL | Cy (1000 mg/m2 ) |

(adapted from Fujiwara, Pharmaceuticals (2014), 7: 1049 -1068)

According to a specific embodiment, the malignant disease is a leukemia, a lymphoma, a myeloma (e.g. multiple myeloma), a melanoma, a sarcoma, a neuroblastoma, a colon cancer, a colorectal cancer, a breast cancer, an ovarian cancer, an esophageal cancer, a synovial cell cancer and a pancreatic cancer.

According to a specific embodiment, the disease includes, but is not limited to, leukemia [e.g., acute lymphatic, acute lymphoblastic leukemia (ALL), acute lymphoblastic pre-B cell, acute lymphoblastic T cell leukemia, acute - megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, acute myelocytic leukemia (AML) or chronic myelocytic leukemia (CML), hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia, acute nonlymphoblastic leukemia (ANLL), T-cell acute lymphocytic leukemia (T-ALL) and B-cell chronic lymphocytic leukemia (B-CLL)], lymphoma (e.g., Hodgkin’s disease, non-Hodgkin’s lymphoma, B cell, Burkitt, cutaneous T cell, histiocytic, lymphoblastic, T cell, thymic).

### Non-malignant Hematologic Disease

Examples of non-malignant hematologic diseases include, but are not limited to, anemia, bone marrow disorders, deep vein thrombosis/pulmonary embolism, diamond blackfan anemia, hemochromatosis, hemophilia, immune hematologic disorders, iron metabolism disorders, sickle cell disease, thalassemia, thrombocytopenia, Von Willebrand disease, severe combined immunodeficiency syndromes (SCID), adenosine deaminase (ADA), aplastic anemia, and other congenital or genetically-determined hematopoietic abnormalities.

### Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Specific types of viral pathogens causing infectious diseases treatable according to the teachings of the present invention include, but are not limited to, retroviruses, circoviruses, parvoviruses, papovaviruses, adenoviruses, herpesviruses, iridoviruses, poxviruses, hepadnaviruses, picornaviruses, caliciviruses, togaviruses, flaviviruses, reoviruses, orthomyxoviruses, paramyxoviruses, rhabdoviruses, bunyaviruses, coronaviruses, arenaviruses, and filoviruses.

Specific examples of viral infections which may be treated according to the teachings of the present invention include, but are not limited to, those caused by human immunodeficiency virus (HIV)-induced acquired immunodeficiency syndrome (AIDS), influenza, rhinoviral infection, viral meningitis, Epstein-Barr virus (EBV) infection, hepatitis A, B or C virus infection, measles, papilloma virus infection/warts, cytomegalovirus (CMV) infection, COVID-19 infection, Herpes simplex virus infection, yellow fever, Ebola virus infection, rabies, Adenovirus (Adv), cold viruses, flu viruses, Japanese encephalitis, polio, respiratory syncytial, rubella, smallpox, varicella zoster, rotavirus, West Nile virus and zika virus.

According to a specific embodiment, the viral disease is caused by a virus selected from the group consisting of Epstein-Barr virus (EBV), cytomegalovirus (CMV), BK Virus, Adenovirus (Adv), severe acute respiratory syndrome (SARS), severe acute respiratory syndrome coronavirus 2 (SARS-CoV-2), immunodeficiency virus (HIV), influenza, Cytomegalovirus (CMV), T-cell leukemia virus type 1 (TAX), hepatitis C virus (HCV) or hepatitis B virus (HBV).

Specific examples of bacterial infections which may be treated according to the teachings of the present invention include, but are not limited to, those caused by anthrax; gram-negative bacilli, chlamydia, diptheria, haemophilus influenza, Helicobacter pylori, malaria, Mycobacterium tuberculosis, pertussis toxin, pneumococcus, rickettsiae, staphylococcus, streptococcus and tetanus.

Specific examples of superbug infections (e.g. multi-drug resistant bacteria) which may be treated according to the teachings of the present invention include, but are not limited to, those caused by Enterococcus faecium, Clostridium difficile, Acinetobacter baumannii, Pseudomonas aeruginosa, and Enterobacteriaceae (including Escherichia coli, Klebsiella pneumoniae, Enterobacter spp.).

Specific examples of fungal infections which may be treated according to the teachings of the present invention include, but are not limited to, those caused by candida, coccidiodes, cryptococcus, histoplasma, leishmania, plasmodium, protozoa, parasites, schistosomae, tinea, toxoplasma, and trypanosoma cruzi.

### Graft Rejection Diseases

According to other embodiment, the disease is associated with transplantation of a graft. Examples of diseases associated with transplantation of a graft include, but are not limited to, graft rejection, chronic graft rejection, subacute graft rejection, hyperacute graft rejection, acute graft rejection, allograft rejection, xenograft rejection and graft-versus-host disease (GVHD).

The genetically modified veto cells of the invention are typically used for non-syngeneic applications. Therefore, the memory T cells or PBMC (i.e. used to generate the genetically modified veto cells) are typically allogeneic with respect to a subject (e.g. from an allogeneic donor). Likewise, in cases in which xenogeneic applications may be beneficial, the memory T cells or PBMC used may be of a xenogeneic origin as discussed below. However, in cases in which a syngeneic applications may be beneficial, the memory T cells or PBMC (i.e. used to generate the genetically modified veto cells) may be autologous with respect to a subject (e.g. from the subject). Such determinations are well within the capability of one of skill in the art, especially in view of the disclosure provided.

As used herein, the term “syngeneic” cells refer to cells which are essentially genetically identical with the subject or essentially all lymphocytes of the subject. Examples of syngeneic cells include cells derived from the subject (also referred to in the art as “autologous”), from a clone of the subject, or from an identical twin of the subject.

As used herein, the term “non-syngeneic” cells refer to cells which are not essentially genetically identical with the subject or essentially all lymphocytes of the subject, such as allogeneic cells or xenogeneic cells.

As used herein, the term “allogeneic” refers to cells which are derived from a donor who is of the same species as the subject, but which is substantially non-clonal with the subject. Typically, outbred, non-zygotic twin mammals of the same species are allogeneic with each other. It will be appreciated that an allogeneic cell may be HLA identical, partially HLA identical or HLA non-identical (i.e. displaying one or more disparate HLA determinant) with respect to the subject.

As used herein, the term “xenogeneic” refers to a cell which substantially expresses antigens of a different species relative to the species of a substantial proportion of the lymphocytes of the subject. Typically, outbred mammals of different species are xenogeneic with each other.

The present invention envisages that xenogeneic cells are derived from a variety of species. Thus, according to one embodiment, the cells may be derived from any mammal. Suitable species origins for the cells comprise the major domesticated or livestock animals and primates. Such animals include, but are not limited to, porcines (e.g. pig), bovines (e.g., cow), equines (e.g., horse), ovines (e.g., goat, sheep), felines (e.g., Felis domestica), canines (e.g., Canis domestica), rodents (e.g., mouse, rat, rabbit, guinea pig, gerbil, hamster), and primates (e.g., chimpanzee, rhesus monkey, macaque monkey, marmoset).

Cells of xenogeneic origin (e.g. porcine origin) are preferably obtained from a source which is known to be free of zoonoses, such as porcine endogenous retroviruses. Similarly, human-derived cells or tissues are preferably obtained from substantially pathogen-free sources.

According to one embodiment, the cells are non-syngeneic with the subject.

According to one embodiment, the cells are allogeneic with the subject.

According to one embodiment, the cells are syngeneic with the subject (e.g. autologous).

According to an embodiment of the present invention, the subject is a human being and the cells are from a human origin (e.g. non-syngeneic).

Thus, the source of the memory T cells or PBMCs (i.e. used to obtain the genetically modified veto cells) will be determined with respect to the intended use of the cells (see further details hereinbelow) and is well within the capability of one skilled in the art, especially in light of the detailed disclosure provided herein.

As discussed above, the veto Tcm cells of the invention are endowed with tolerance-inducing activity. Thus, the use of genetically modified veto cells is especially beneficial in situations in which there is a need to eliminate graft rejection and overcome graft versus host disease (GvHD). Accordingly, the veto Tcm cells of the present invention may be used as adjuvant therapy for a cell or tissue transplant.

As the Tcm cells of the present invention are also endowed with anti-disease activity, the method of the present invention can furthermore be advantageously applied towards treating a disease in a subject while concomitantly facilitating engraftment of a transplant of cells or tissues. Thus, the genetically modified veto cells may be advantageously used for killing diseased cells (e.g. cancer cells such as during relapse or residual cells) and preventing infections, such as in transplantation of allogeneic or xenogeneic cells or tissues.

Thus, as mentioned above, the genetically modified veto cells of the invention are endowed with anti-disease activity (e.g. anti-viral activity and/or anti-tumor activity) and are therefore beneficial in situations in which a subject, e.g. transplanted subject, has a disease or condition (e.g. malignant disease, viral infection, bacterial infection, fungal infection), pre- or post-transplantation (e.g. before immune reconstitution is established), while concomitantly eliminating graft rejection and overcome graft versus host disease (GvHD).

According to one embodiment, the method further comprises transplanting a cell or tissue transplant into the subject.

According to one embodiment there is provided a method of treating a subject in need of a cell or tissue transplantation, the method comprising: (a) transplanting a cell or tissue transplant into the subject; and (b) administering to the subject an effective amount of the isolated population of genetically modified veto cells of some embodiments of the invention, thereby treating the subject in need of the cell or tissue transplantation.

According to one embodiment, there is provided a therapeutically effective amount of the isolated population of genetically modified veto cells of some embodiments of the invention for use as an adjuvant treatment for a cell or tissue transplant into a subject, wherein the subject is in need of a cell or tissue transplantation.

According to one embodiment, transplanting is affected concomitantly with, prior to, or following administering of the genetically modified veto cells.

As used herein, the phrase “cell or tissue transplantation” refers to a bodily cell (e.g. a single cell or a group of cells) or tissue (e.g. solid tissues/organs or soft tissues, which may be transplanted in full or in part). Exemplary tissues or organs which may be transplanted according to the present teachings include, but are not limited to, liver, pancreas, spleen, kidney, heart, lung, skin, intestine and lymphoid/hematopoietic tissues (e.g. lymph node, Peyer’s patches thymus or bone marrow). Exemplary cells which may be transplanted according to the present teachings include, but are not limited to, immature hematopoietic cells, including stem cells, cardiac cells, hepatic cells, pancreatic cells, spleen cells, pulmonary cells, brain cells, nephric cells, intestine/gut cells, ovarian cells, skin cells, (e.g. isolated population of any of these cells). Furthermore, the present invention also contemplates transplantation of whole organs, such as for example, kidney, heart, liver or skin.

Depending on the application, the method may be affected using a cell or tissue which is syngeneic or non-syngeneic with the subject.

According to an embodiment of the present invention, both the subject and the cell or tissue donor are humans.

Depending on the application and available sources, the cells or tissues of the present invention may be obtained from a prenatal organism, postnatal organism, an adult or a cadaver donor. Moreover, depending on the application needed the cells or tissues may be naïve or genetically modified. Such determinations are well within the ability of one of ordinary skill in the art.

Any method known in the art may be employed to obtain a cell or tissue (e.g. for transplantation).

Transplanting the cell or tissue into the subject may be affected in numerous ways, depending on various parameters, such as, for example, the cell or tissue type; the type, stage or severity of the recipient’s disease (e.g. organ failure); the physical or physiological parameters specific to the subject; and/or the desired therapeutic outcome.

Transplanting a cell or tissue transplant of the present invention may be affected by transplanting the cell or tissue transplant into any one of various anatomical locations, depending on the application. The cell or tissue transplant may be transplanted into a homotopic anatomical location (a normal anatomical location for the transplant), or into an ectopic anatomical location (an abnormal anatomical location for the transplant). Depending on the application, the cell or tissue transplant may be advantageously implanted under the renal capsule, or into the kidney, the testicular fat, the sub cutis, the omentum, the portal vein, the liver, the spleen, the heart cavity, the heart, the chest cavity, the lung, the skin, the pancreas and/or the intra abdominal space.

For example, a liver tissue according to the present teachings may be transplanted into the liver, the portal vein, the renal capsule, the sub-cutis, the omentum, the spleen, and the intra-abdominal space. Transplantation of a liver into various anatomical locations such as these is commonly practiced in the art to treat diseases amenable to treatment via hepatic transplantation (e.g. hepatic failure). Similarly, transplanting a pancreatic tissue according to the present invention may be advantageously affected by transplanting the tissue into the portal vein, the liver, the pancreas, the testicular fat, the sub-cutis, the omentum, an intestinal loop (the subserosa of a U loop of the small intestine) and/or the intra-abdominal space. Transplantation of pancreatic tissue may be used to treat diseases amenable to treatment via pancreatic transplantation (e.g. diabetes). Likewise, transplantation of tissues such as a kidney, a heart, a lung or skin tissue may be carried out into any anatomical location described above for the purpose of treating recipients suffering from, for example, renal failure, heart failure, lung failure or skin damage (e. g., burns). In cases in which isolated cells are transplanted, such cells may be administered via, for example, an intravenous route, an intratracheal route, an intraperitoneal route, or an intranasal route.

The method of the present invention may also be used, for example, for treating a recipient suffering from a disease requiring immature hematopoietic cell transplantation.

In the latter case, immature autologous, allogeneic or xenogeneic hematopoietic cells (including stem cells) which can be derived, for example, from bone marrow, mobilized peripheral blood (by for example leukapheresis), fetal liver, yolk sac and/or cord blood of the donor can be transplanted to a recipient suffering from a disease. According to one embodiment, the immature hematopoietic cells are T-cell depleted CD34+ immature hematopoietic cells.

It will be appreciated that the immature autologous or allogeneic hematopoietic cells of the present invention may be transplanted into a recipient using any method known in the art for cell transplantation, such as but not limited to, cell infusion (e.g. I.V.) or via an intraperitoneal route.

Optionally, when transplanting a cell or tissue transplant of the present invention into a subject having a defective organ, it may be advantageous to first at least partially remove the failed organ from the subject so as to enable optimal development of the transplant, and structural/functional integration thereof with the anatomy/physiology of the subject.

According to one embodiment, the cell or tissue transplant is derived from an allogeneic donor. According to one embodiment, the cell or tissue transplant is derived from an HLA identical allogeneic donor or from an HLA non-identical allogeneic donor. According to one embodiment, the cell or tissue transplant is derived from a xenogeneic donor.

According to one embodiment, the cell or tissue transplant and the genetically modified veto Tcm cells are derived from the same (e.g. non-syngeneic) donor.

According to one embodiment, the cell or tissue transplant and the genetically modified veto cells are derived from different (e.g. non-syngeneic) donors. Accordingly, the cell or tissue transplant may be non-syngeneic with the genetically modified veto Tcm cells.

According to one embodiment, the immature hematopoietic cells and the genetically modified veto Tcm cells are derived from the same (e.g. non-syngeneic) donor.

According to one embodiment, the immature hematopoietic cells and the genetically modified veto Tcm cells are derived from different (e.g. non-syngeneic) donors. Accordingly, the immature hematopoietic cells may be non-syngeneic with the genetically modified veto Tcm cells.

The method of the present invention also envisions co-transplantation of several organs (e.g. cardiac and pulmonary tissues) in case the subject may be beneficially affected by such a procedure.

According to one embodiment, the co-transplantation comprises transplantation of immature hematopoietic cells and a solid tissue/organ or a number of solid organs/tissues.

According to one embodiment, the immature hematopoietic cells and the solid organ or obtained from the same donor.

According to another embodiment, the immature hematopoietic cells and the solid organ/tissue or organs/tissue are obtained from different (e.g. non-syngeneic) donors.

According to one embodiment, the immature hematopoietic cells are transplanted prior to, concomitantly with, or following the transplantation of the solid organ.

According to an embodiment, hematopoietic chimerism is first induced in the subject by transplantation of immature hematopoietic cells in conjunction with the genetically modified veto Tcm cells of some embodiments the present invention, leading to tolerance of other tissues/organs transplanted from the same donor.

According to an embodiment, the genetically modified veto Tcm cells of the present invention are used per se for reduction of rejection of transplanted tissues/organs transplanted from the same donor.

According to an embodiment, the genetically modified veto Tcm cells of the present invention are used per se for killing residual cancer cells or for disease relapse.

According to an embodiment, the genetically modified veto Tcm cells of the present invention are used per se for the treatment of infectious disease (e.g. viral disease).

Following transplantation of the cell or tissue transplant into the subject according to the present teachings, it is advisable, according to standard medical practice, to monitor the growth functionality and immuno-compatibility of the organ according to any one of various standard art techniques. For example, the functionality of a pancreatic tissue transplant may be monitored following transplantation by standard pancreas function tests (e.g. analysis of serum levels of insulin). Likewise, a liver tissue transplant may be monitored following transplantation by standard liver function tests (e.g. analysis of serum levels of albumin, total protein, ALT, AST, and bilirubin, and analysis of blood-clotting time). Structural development of the cells or tissues may be monitored via computerized tomography, or ultrasound imaging.

Depending on the transplantation context, in order to facilitate engraftment of the cell or tissue transplant, the method may further advantageously comprise conditioning the subject under sublethal, lethal or supralethal conditions prior to the transplanting.

As used herein, the terms “sublethal”, “lethal”, and “supralethal”, when relating to conditioning of subjects of the present invention, refer to myelotoxic and/or lymphocytotoxic treatments which, when applied to a representative population of the subjects, respectively, are typically: non-lethal to essentially all members of the population; lethal to some but not all members of the population; or lethal to essentially all members of the population under normal conditions of sterility.

According to some embodiments of the invention, the sublethal, lethal or supralethal conditioning comprises a total body irradiation (TBI), total lymphoid irradiation (TLI, i.e. exposure of all lymph nodes, the thymus, and spleen), partial body irradiation (e.g. specific exposure of the lungs, kidney, brain etc.), myeloablative conditioning and/or non-myeloablative conditioning, e.g. with different combinations including, but not limited to, co-stimulatory blockade, chemotherapeutic agent and/or antibody immunotherapy. According to some embodiments of the invention, the conditioning comprises a combination of any of the above described conditioning protocols (e.g. chemotherapeutic agent and TBI, co-stimulatory blockade and chemotherapeutic agent, antibody immunotherapy and chemotherapeutic agent, etc.).

According to one embodiment, the TBI comprises a single or fractionated irradiation dose within the range of 0.5-1 Gy, 0.5-1.5 Gy, 0.5-2.5 Gy, 0.5-5 Gy, 0.5-7.5 Gy, 0.5-10 Gy, 0.5-15 Gy, 1-1.5 Gy, 1-2 Gy, 1-2.5 Gy, 1-3 Gy, 1-3.5 Gy, 1-4 Gy, 1-4.5 Gy, 1-1.5 Gy, 1-7.5 Gy, 1-10 Gy, 2-3 Gy, 2-4 Gy, 2-5 Gy, 2-6 Gy, 2-7 Gy, 2-8 Gy, 2-9 Gy, 2-10 Gy, 3-4 Gy, 3-5 Gy, 3-6 Gy, 3-7 Gy, 3-8 Gy, 3-9 Gy, 3-10 Gy, 4-5 Gy, 4-6 Gy, 4-7 Gy, 4-8 Gy, 4-9 Gy, 4-10 Gy, 5-6 Gy, 5-7 Gy, 5-8 Gy, 5-9 Gy, 5-10 Gy, 6-7 Gy, 6-8 Gy, 6-9 Gy, 6-10 Gy, 7-8 Gy, 7-9 Gy, 7-10 Gy, 8-9 Gy, 8-10 Gy, 10-12 Gy or 10-15 Gy.

According to a specific embodiment, the TBI comprises a single or fractionated irradiation dose within the range of 1-7.5 Gy.

According to one embodiment, the conditioning is affected by conditioning the subject under supralethal conditions, such as under myeloablative conditions.

Alternatively, the conditioning may be affected by conditioning the subject under lethal or sublethal conditions, such as by conditioning the subject under myeloreductive conditions or non-myeloablative conditions.

According to one embodiment, the conditioning is affected by conditioning the subject with a myeloablative drug (e.g. Busulfan and/or Melfaln) or a non-myeloablative drug (e.g. Cyclophosphamide and/or Fludarabin).

Examples of conditioning agents which may be used to condition the subject include, without limitation, irradiation and pharmacological agents.

Examples of pharmacological agents include myelotoxic drugs, lymphocytotoxic drugs and immunosuppressant drugs (discussed in detail below).

Examples of myelotoxic drugs include, without limitation, busulfan, dimethyl mileran, melphalan and thiotepa.

Additionally or alternatively, the method may further comprise conditioning the subject with an immunosuppressive regimen prior to, concomitantly with, or following transplantation of the cell or tissue transplant.

Examples of suitable types of immunosuppressive regimens include administration of immunosuppressive drugs and/or immunosuppressive irradiation.

Ample guidance for selecting and administering suitable immunosuppressive regimens for transplantation is provided in the literature of the art (for example, refer to: Kirkpatrick CH. and Rowlands DT Jr., 1992. JAMA. 268, 2952; Higgins RM. et al., 1996. Lancet 348, 1208; Suthanthiran M. and Strom TB., 1996. New Engl. J. Med. 331, 365; Midthun DE. et al., 1997. Mayo Clin Proc. 72, 175; Morrison VA. et al., 1994. Am J Med. 97, 14; Hanto DW., 1995. Annu Rev Med. 46, 381; Senderowicz AM. et al., 1997. Ann Intern Med. 126, 882; Vincenti F. et al., 1998. New Engl. J. Med. 338, 161; Dantal J. et al. 1998. Lancet 351, 623).

Preferably, the immunosuppressive regimen consists of administering at least one immunosuppressant agent to the subject.

Examples of immunosuppressive agents include, but are not limited to, Tacrolimus (also referred to as FK-506 or fujimycin, trade names: Prograf, Advagraf, Protopic), Mycophenolate Mofetil, Mycophenolate Sodium, Prednisone, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE), etanercept, TNF.alpha. blockers, a biological agent that targets an inflammatory cytokine, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors, tramadol, rapamycin (sirolimus) and rapamycin analogs (such as CCI-779, RAD001, AP23573). These agents may be administered individually or in combination.

Regardless of the transplant type, to avoid graft rejection and graft versus host disease, the method of the present invention utilizes the novel genetically modified veto Tcm cells (as described in detail hereinabove).

According to the method of the present invention, these genetically modified veto Tcm cells are administered either concomitantly with, prior to, or following the transplantation of the cell or tissue transplant.

The genetically modified veto Tcm cells may be administered via any method known in the art for cell transplantation, such as but not limited to, cell infusion (e.g. intravenous) or via an intraperitoneal route, as discussed above.

In order to enhance the anti-disease activity of the genetically modified veto Tcm cells, it is beneficial to select an antigen or antigens associated with the disease to be treated and to generate antigen specific Tcm cells for treatment.

Thus, according to one embodiment, there is provided a method of treating a disease in a subject in need thereof, the method comprising:
(a) analyzing a biological sample of a subject for the presence of an antigen or antigens associated with the disease;
(b) generating genetically modified veto cells according to the method of some embodiments of the invention towards the antigen or antigens associated with the disease; and
(c) administering to the subject a therapeutically effective amount of the genetically modified veto cells of step (b), thereby treating the disease in the subject.

As used herein “a biological sample” refers to a sample of fluid or tissue sample derived from a subject. Examples of “biological samples” include but are not limited to whole blood, serum, plasma, cerebrospinal fluid, urine, lymph fluids, tissue biopsy, and various external secretions of the respiratory, intestinal and genitourinary tracts, tears, saliva, milk as well as white blood cells, tissues, cell culture e.g., primary culture.

Methods of obtaining such biological samples are known in the art including but not limited to standard blood retrieval procedures, urine collection, and lumbar puncture.

Determining the presence of an antigen or antigens in a biological sample can be carried out using any method known in the art, e.g. by serology (testing for the presence of a pathogen), bacterial culture, bacterial susceptibility testing, tests for fungi, viruses, mycobacteria (AFB testing) and/or parasites, electrophoresis, enzyme linked immunosorbent assay (ELISA), western blot analysis and Fluorescence activated cell sorting (FACS).

Once analysis is made, the antigen or antigens are selected and genetically modified veto Tcm cells are generated from a population of memory CD8⁺ T cells, as discussed above, using the antigen or antigens specific for the disease (e.g. tumor antigens, viral antigens, bacterial antigens, etc.) and are administered to the subject for treatment.

As used herein the term “about” refers to ± 10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 1 is expressed in a DNA sequence format (e.g., reciting T for thymine), but it can refer to either a DNA sequence that corresponds to an CAR retroviral vector nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization - A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### GENERAL MATERIALS AND EXPERIMENTAL PROCEDURES

### Generation of Central Memory Anti-viral CD8⁺ veto cells (Tcm)

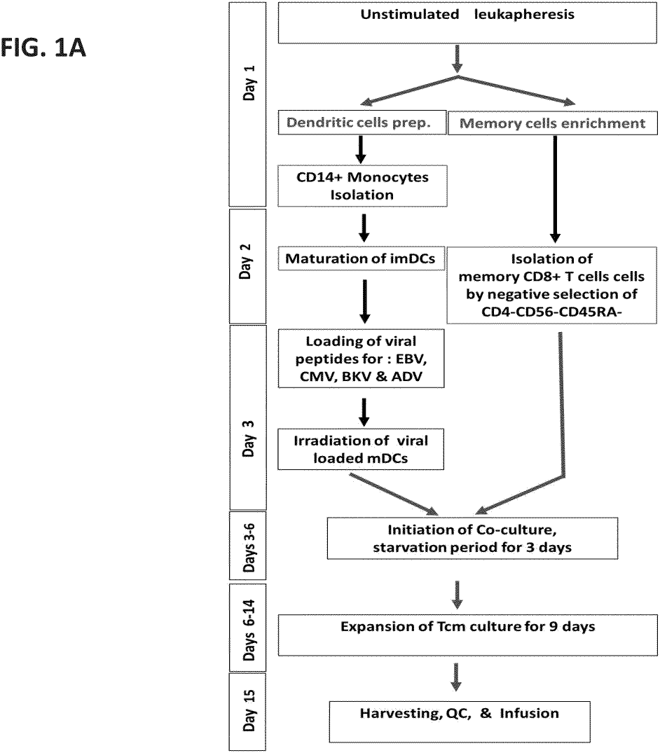

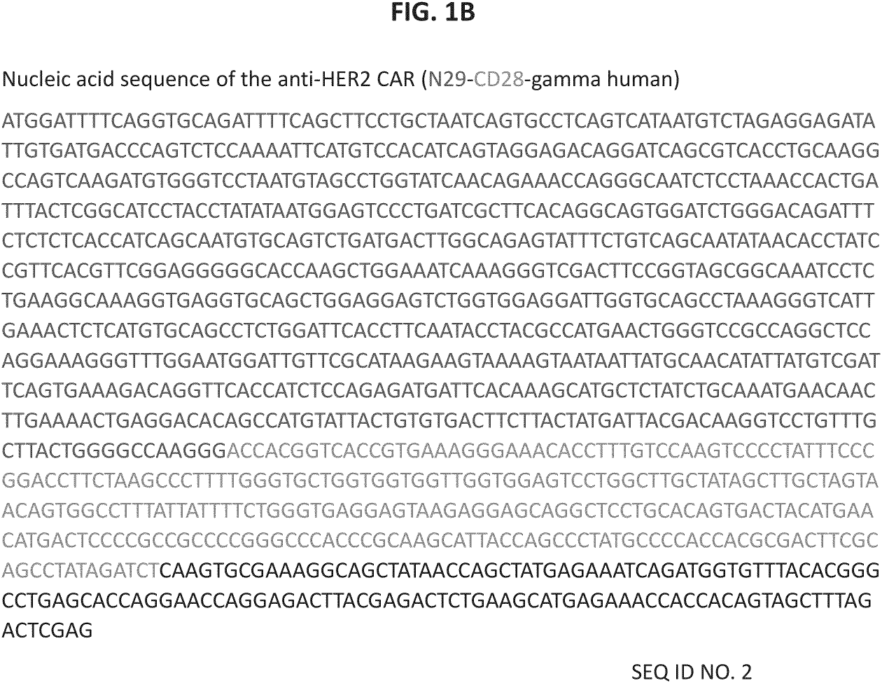

As can be seen in FIG. 1A, mononuclear cells obtained after leukapheresis and ficol fractionation are depleted of CD4⁺ and CD45RA⁺ T cells and of CD56⁺ NK cells by magnetic beads fractionation. The remaining cells (enriched for CD8⁺CD45RA⁻ memory CD8⁺ T cells) were co-cultured with dendritic cells (DCs) obtained from the same donor, pulsed with different viral peptides of four viruses including CMV, EBV, Adeno and BK virus and irradiated by 30 Gy . During the initial 3 days of culture only IL-21 was used in the culture medium and thereafter IL-15 and IL-7 were also added. Central memory T cells (Tcm) were harvested after 12 days of culture.

### Construction of CAR Retroviral Vectors

The N29 monoclonal antibody against human ErbB-2 was used as a source of the scFv, for construction of the N29 CAR [Stancovski 1991]. N29 was ligated to the CD28 co-stimulation domain and the γITAM subunit domain. The scFv N29 CAR was cloned into the pBullet retroviral vector (as illustrated in FIG. 1B and set forth in SEQ ID NO: 2).

### Preparation of Packaging Cells

293T cells were transfected using Ca₂PO₄ with GAG-POL pCL-Ampho retroviral envelop and the CAR-encoding MSGV-1/pBullet vectors. Retroviral supernatant was collected and used to stably transduce the amphotropic PG13 packaging cells. Infected cells were sorted to enrich CAR-expressing cells. Packaging cells with the highest levels of CAR expression and infectious titer were collected, re-grown, and frozen in aliquots at -80° C.

### T-cell Transduction

Retroviral transduction of T cells was performed as described previously [Eshhar Z. et al., Functional expression of chimeric receptor genes in human T cells. J Immunol Methods (2001) 248:67-76]. Briefly, peripheral human blood lymphocytes (PBL) were isolated from the blood of healthy human donors by density gradient centrifugation on Ficoll-Paque (Axis-shield, Oslo, Norway). Human anti-viral veto cells were prepared as described above. Control PBMCs were activated in non-tissue culture-treated 6-well plates, pre-coated with both purified anti-human CD3 and purified anti-human CD28 for 48 hours at 37° C. Both veto cells or the activated PBMCs were subjected to two consecutive retroviral transductions in RetroNectin (Takara) pre-coated non-tissue culture-treated 6-well plates supplemented with human IL-2 (100 IU/mL) or a cytokines cocktail including IL-21, IL-15 and IL-7 for VETO, respectively. After transduction, activated PBMCs were cultured in the presence of 350 IU/mL IL-2 and VETO in the presence of the cytokine cocktail (as discussed above). Transduction efficiency was monitored by flow cytometry on day 6 of transduction. Non-infected cells were included as T cell controls.

### Flow Cytometry Analysis

Analysis of cell surface expression of N29 specific CAR receptors was based on detection of their extracellular scFv moieties. A 2-step staining protocol was used, which included an initial incubation of CAR-transduced T cells with anti-N29-biotin antibody followed by incubation with streptavidin-APC. Expression of N29 was also estimated based on green fluorescence protein (GFP) fluorescence (the N29 CAR gene sequence is followed by the combined sequences of an internal ribosome entry site (IRES) and GFP in the expression vector). Staining and cell washes were done in FACS Buffer (containing 2% fetal bovine serum, 0.05% sodium azide, and 2 mM EDTA pH=8 in PBS).

### In-vitro Assessment of Reaction Target by the Lymphocyte CAR-T Cells and VETO CAR-T cells

Lymphocyte CAR-T cells or VETO CAR-T cells and their corresponding non-infected control cells, were incubated for 24 hours with target SKOV cells at a 1:2 target: effector (T:E) ratio. Cell-free growth medium was collected and analyzed for IFN-γ secretion by ELISA using a human IFN-γ ELISA kit, according to the manufacturer’s instructions (R&D systems).

### CAR-T Cell -mediated Cell Killing

The cytotoxicity of transfected T cells was determined by a methylene blue staining-based assay. N29 CAR-T lymphocytes, N29 VETO CAR-T cells or non-infected cells were incubated with target cells in a 96-well plate at T:E ratios of 1:4, 1:2, 1:1, 1:0.5 and 1:0.25. After 16 hours, the plate was washed with PBS to remove T cells and dead target cells. Live cells that remained attached to the culture plate were fixed with 4% formaldehyde for 2 hours at room temperature (RT), washed twice with 0.1 M sodium borate pH 8.5 and stained with 0.5% methylene blue (Sigma Aldrich) diluted in 0.1 M sodium borate for 15 minutes at RT. Cells were then washed under tap water to remove excess methylene blue solution. 0.1 M HCL was added prior to analysis. Absorbance at 620 nm was read on a Multiskan FC ELISA reader (Thermo Fisher Scientific). Percent killing was calculated according to the following equation: 100-(absorbance of target cell incubated with CAR-T cells/absorbance of target cell incubated without CAR-T cells x 100).

### Estimate of Veto Activity Before and After Transfection

The assessment of veto activity consisted of 3 steps:

The first stage was a bulk culture of host derived PBMC (effectors) with donor derived irradiated or 3^{rd} party irradiated stimulators in the presence of donor type veto cells in different ratios (1:1 or 1:5) for 5 days at 37° C. 5% CO₂ in Cellgro medium (Cellgenix) supplemented with Pen/Strep and 5% HS.

Next, limiting dilution assay (LDA) from the bulk culture was established for amplification and titration of the host anti-donor end point signal. Specifically, cells from each bulk culture were fractionated on Ficoll-Paque Plus gradient (Amersham Pharmacia Biotech, Uppsala, Sweden), washed in PBS and re-suspended in RPMI-1640 culture medium supplemented with 5% HS L-glutamine, pen/strep, HEPES, Na-pyruvate, NEAA and 2β-mercaptoethanol. The cells from each culture were then counted and brought to a concentration of 0.4 x 10⁶ cell/ml. Sixteen replicates containing 100 µL of responder cells from each culture were then double diluted so as to obtain different responder cell number per well (40000, 20000, 10000, 5000, 2500, 1250, 625, 312, 156, 78, 39, 19, 9, 4, 2, 0) in U shape-bottomed, 96-well plates (Nunc, Roskilde, Denmark). 100 µL containing 10⁵ irradiated stimulators (donor type-specific or 3^{rd} party type-non-specific) were then added to each well with 10 U/ml IL-2. The plates were incubated for 7 days 37° C. 5% CO₂.

Lastly, at the end of the culture, killing efficacy of target cells was determined by transferring 100 µL (including responder cells) from each well to 96 wells V shape plate (Greiner Bio one Italy) and incubating for 5 hours (37° C. 5% CO₂) with 5 x 10³ blasts (target cells) from the specific donor or a third party (non-specific donor) that had been stimulated with concanavalin A (Sigma, St Louis, MO) and radioactively labeled with Easy-Tag ³⁵S Met (Perkin Elmer). Of note, the specific donor was blast generated from the same donor from which the CAR veto were generated, while the non-specific donor was blast generated from another donor that has different HLA typing set from that of the donor of CAR veto cells (i.e. different HLA allelles). Following the incubation, the plates were centrifuged and 50 µL of supernatant (not including cells) was transferred to counting 96 plates (Optiplate, Perkin Elmer) and 150 µL of scintillation fluid (microscint 40) was added to each well. Each plate was covered with plastic stick and incubated overnight in room temperature. Luminescence resulting from radioactive material decay in the supernatants was measured by TopCount Luminescence counter (Packard Instrument Co., Inc). The release of ³⁵S Met by target cells cultured in medium alone was defined as spontaneous release. In addition, cells lysis with 1% sodium dodecyl sulfate was defined as total release. To calculate the Frequency of host CTL-p from the limiting-dilution culture readout [Taswell. C, Limiting dilution assays for the determination of immunocompetent cell frequencies. I. Data analysis. Journal of Immunol (1981) 126(4): 1614-1619], the following equation was used: lnY= - fx + lna (which represents the zero-order term of the Poisson distribution), in which y is the percentage of nonresponding cultures, x is the number of responding cells per culture, f is the frequency of responding precursors, and a is the y-intercept theoretically equal to 100%. Microwell cultures were considered positive for a cytolytic response when values exceeded the mean spontaneous release value by at least 3 SDs of the mean. The percentage of responding cultures was defined by calculating the percentage of positive cultures. The CTL-p frequency (f) were determined from the slope of the line drawn by using linear regression analysis of the data.

### Anti-viral Activity of Tcm Cells as Determined by Intracellular Staining

Veto-Tcm cells were co-cultured with the respective viral peptide mix (EBV, CMV, Adeno, BKV) in the presence of Brefeldin A (eBioscience) at 37° C., 5% CO₂ for 6 hrs. Cells were fixed, permeabilized (Invitrogen Fix & Perm set), and immunostained for CD45, CD3, CD8, IFN-γ, and TNF-α (BD). Positive control included TCR-independent stimulation with PMA/Ionomycin. Cells were gated on a CD45⁺/FSC lymphocyte gate and CD3⁺CD8⁺.

### Example 1

### Defining Alloreactivity in Anti-viral CD8⁺ Veto Cell Preparation Generated from Memory T Cells

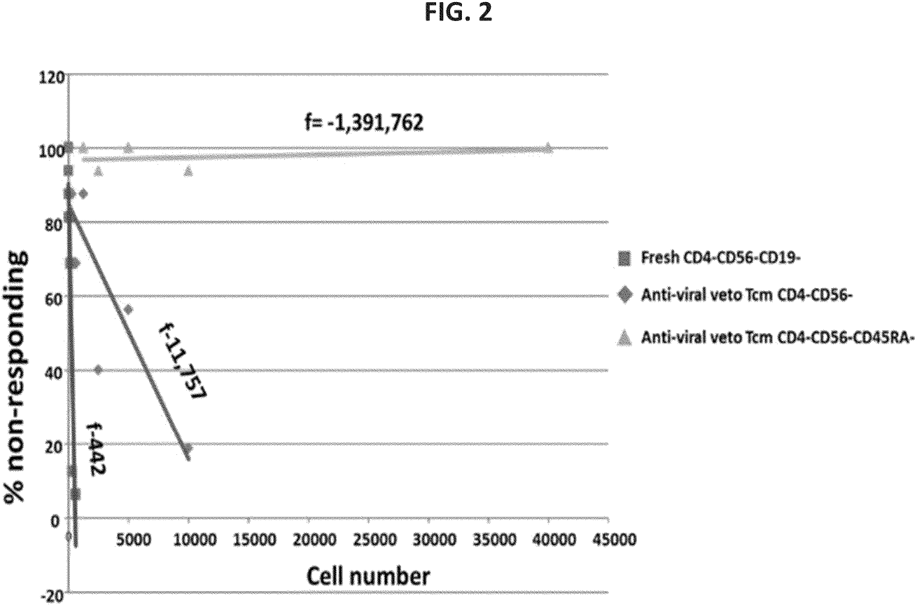

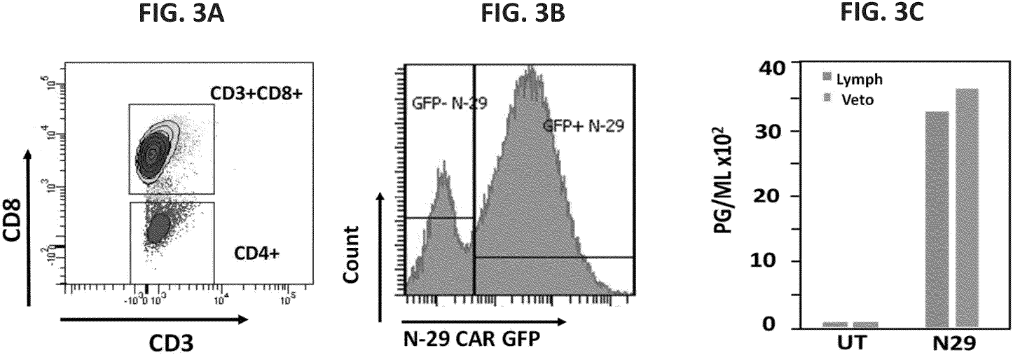

The present inventors generated central memory CD8⁺ veto cells from the T cell memory pool of a donor using stimulation against donor dendritic cells (DCs) pulsed with viral peptides (EBV, CMV, Adenovirus and BK virus). Specifically, CD8⁺ memory T cells were isolated from normal PBMC by depletion of CD4⁺, CD56⁺ and CD45RA⁺ cells and were co-cultured with DCs loaded with the viral peptides (as described in FIG. 1A). The resultant cells’ anti-host reactivity was then tested using a limiting dilution analysis (LDA) killing assay demonstrating that these cells do not exert any anti-host reactivity (FIG. 2).

### Example 2

### Anti-tumor Activity of Human VETO CAR-CD8⁺ T Cells

It is well established that allogenic hematopoietic stem cell transplantation (HSCT) in patients with hematological malignancies enhances Graft versus Leukemia (GvL) effect as compared to results with autologous HSCT (by virtue of the new established immune system derived from the allogenic donor). However, the former is associated with higher risk for transplant related mortality (TRM). The new approach described herein - making use of low conditioning combined with megadose T cell depleted HSCT plus anti-viral veto cells - could lower significantly the risk for TRM while preserving its GvL activity. Considering that immune reconstitution post-transplant requires at least a few months, this approach is suitable largely for patients in remission or with minimal residual disease at the day of transplant. Therefore, combining the attributes of CAR-T cell therapy with allogenic HSCT, two modalities operating via different mechanisms, by using Veto CAR-T cells, could be extremely attractive as it could extend approach to patients with significant residual disease or in relapse.

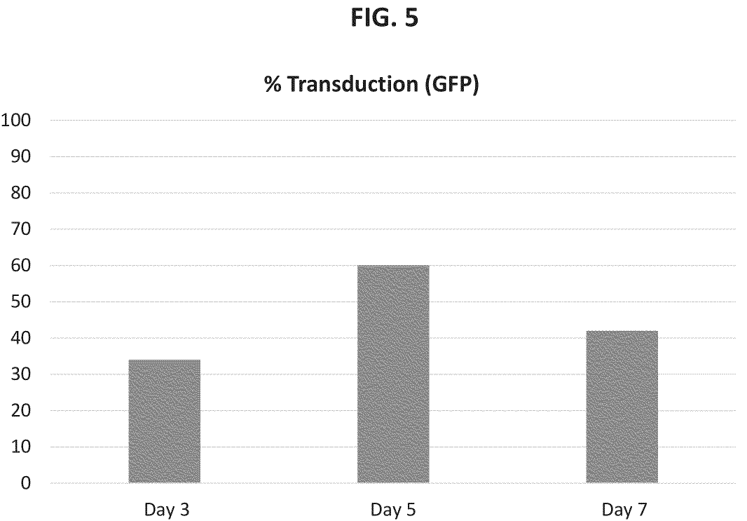

In previous experiments it was demonstrated that genetically modified veto cells expressing a T cell receptor transgene against OVA could induce tolerance by virtue of their veto activity and thereby prolong their survival in mis-matched recipient mice (data not shown). Furthermore, such genetically modified veto cells were able to eliminate OVA expressing melanoma cells, suggesting a proof of concept for the usefulness of veto CAR-T cells (data not shown). The transfection procedure has now been optimized, via a set of in-vitro studies, yielding high levels of CAR expression in human anti-viral CD8⁺ veto cells (described above). Using the current protocol for production of anti-viral central memory veto CD8⁺ T cells involving 9 days of culture, transfection efficacy was evaluated at different time points in culture using a CAR directed against the Her2 antigen as a proof of concept model. The results suggested that optimal transfection was attained on day 5 of culture (FIG. 5). Thus, the veto cell product harvested on day 9 exhibited 92% CD8⁺ T cells (FIG. 3A) of which 72% expressed the transduced anti-Her2 CAR (FIG. 3B) with specific reactivity against Her2 antigen similar to that exhibited in a control experiment with regular transduced T cells (FIG. 3C). Notably, after transfection the cells continued to exhibit veto activity (FIGS. 3D-E).

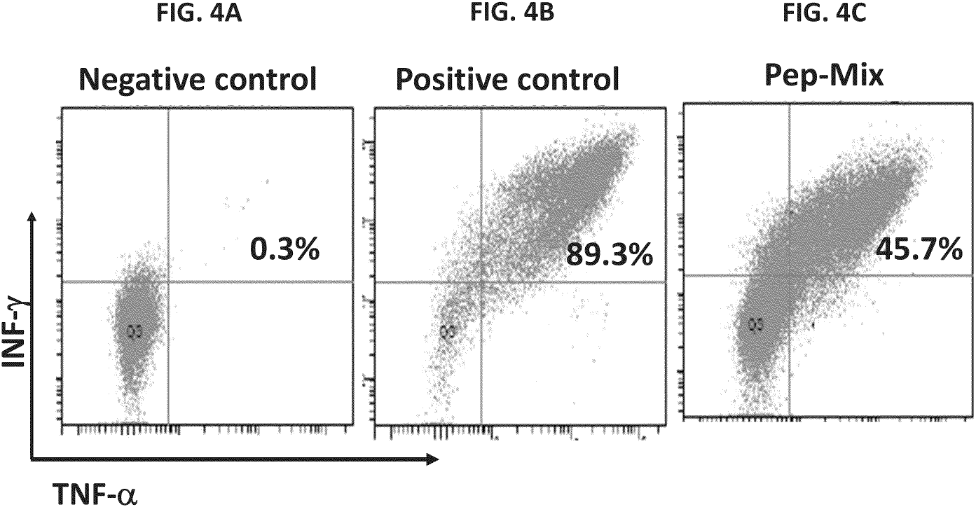

Furthermore, the veto cell product uniquely exhibited anti-viral activity directed against a mix of viral peptides, i.e. EBV, CMV, Adeno, BKV, as illustrated by INF-γ and TNF-α staining (FIGS. 4A-C).

Taken together, two types of clinical applications for these veto CAR-T cells are envisioned:

In the context of haploidentical HSCT, using veto cells from the HSCT donor. Such a donor derived veto CAR-T cell offer three major attributes, namely, (a) enhancement of engraftment, (b) anti-viral activity and (c) graft versus leukemia activity - all to reduce minimal residual disease and prevent leukemia relapse post-transplant.

Outside of the HSCT context, using veto CAR-T cells as an off-the-shelf product for the treatment of patients with hematological malignancies and solid tumors such as those in relapse or having metastasis.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of generating a population of genetically modified veto cells, the method comprising:
(a) providing a population of cells comprising T cells, said T cells comprising at least 40% memory CD8⁺ T cells;
(b) culturing said population of cells comprising T cells with an antigen or antigens under conditions which allow enrichment of tolerance-inducing antigen-specific cells having a central memory T-lymphocyte (Tcm) phenotype, the cells being depleted of graft versus host (GVH) reactivity; and
(c) transducing said cells with a polynucleotide encoding a heterologous cell surface receptor comprising a T cell receptor signaling module,
thereby generating the population of genetically modified veto cells.

2. The method of claim 1, wherein said step (b) is affected concomitantly with step (c).

3. The method of claim 1, wherein step (a) is affected by treating peripheral blood mononuclear cells (PBMCs),
(i) with an agent capable of depleting CD4⁺, CD56⁺ and CD45RA⁺ cells; or
(ii) with an agent capable of selecting CD45RO⁺, CD8⁺ cells,
so as to obtain a population of cells comprising T cells enriched of memory CD8⁺ T cells comprising a CD45RO⁺CD45RA⁻CD8⁺ phenotype.

4. The method of claim 1, wherein said heterologous cell surface receptor comprises a chimeric antigen receptor (CAR) or a transgenic T cell receptor (tg-TCR).

5. The method of claim 4, wherein said CAR comprises at least one co-stimulatory domain and/or at least one signaling domain.

6-8. (canceled)

9. The method of claim 4, wherein said CAR or said tg-TCR binds an antigen selected from the group consisting of a tumor antigen, a viral antigen, a bacterial antigen, a fungal antigen, a protozoa antigen, and a parasite antigen.

10-12. (canceled)

13. The method of claim 1, wherein said memory CD8⁺ T cells are devoid of CD45RA⁺ cells; and/or devoid of CD4⁺ and/or CD56⁺ cells and/or comprise a CD45RO⁺CD45RA⁻CD8⁺ phenotype.

14-15. (canceled)

16. The method of claim 1, wherein said culturing said population of cells comprising T cells with an antigen or antigens under conditions which allow enrichment of tolerance-inducing antigen-specific cells having a Tcm phenotype, is affected by a method comprising:
(a) contacting said population of cells comprising T cells with said antigen or antigens in the presence of IL-21 so as to allow enrichment of antigen reactive cells; and
(b) culturing said cells resulting from step (a) in the presence of IL-21, IL-15 and/or IL-7 so as to allow proliferation of cells comprising said Tcm phenotype.

17. The method of claim 1, wherein said antigen or antigens comprise third-party antigen or antigens.

18-24. (canceled)

25. The method of claim 16, wherein said method is affected ex-vivo.

26. The method of claim 16, wherein said Tcm phenotype comprises a CD3⁺, CD8⁺, CD62L⁺, CD45RA⁻, CD45RO⁺ signature.

27. (canceled)

28. The method of claim 1, wherein said transducing is affected on days 3-7 of culture.

29. (canceled)

30. The method of claim 1, wherein said genetically modified veto cells are endowed with anti-disease activity.

31. The method of claim 1, wherein at least 10% of the veto cells within the population of cells express said heterologous cell surface receptor.

32. An isolated population of genetically modified veto cells obtainable according to the method of claim 1.

33. A pharmaceutical composition comprising the isolated population of genetically modified veto cells of claim 32 and a pharmaceutically active carrier.

34. A method of treating a disease in a subject in need thereof, the method comprising administering to the subject a therapeutically effective amount of the isolated population of genetically modified veto cells of claim 32, thereby treating the subject.

35. (canceled)

36. A method of treating a disease in a subject in need thereof, the method comprising:
(a) analyzing a biological sample of a subject for the presence of an antigen or antigens associated with the disease;
(b) generating genetically modified veto cells according to the method of claim 1 towards said antigen or antigens associated with the disease; and
(c) administering to the subject a therapeutically effective amount of the genetically modified veto cells of step (b), thereby treating the disease in the subject.

37. The method of claim 36, further comprising transplanting a cell or tissue transplant into the subject.

38. (canceled)

39. A method of treating a subject in need of a cell or tissue transplantation, the method comprising:
(a) transplanting a cell or tissue transplant into the subject; and
(b) administering to the subject an effective amount of the isolated population of genetically modified veto cells of claim 32, thereby treating the subject in need of the cell or tissue transplantation.

40. (canceled)

41. The method of claim 39, wherein said transplanting is affected concomitantly with, prior to, or following said administering of said genetically modified veto cells.

42. (canceled)

43. The method of claim 34, wherein the disease is selected from the group consisting of a malignant disease, a viral disease, a bacterial disease, a fungal disease, a protozoa disease, and a parasite disease.

44-45. (canceled)

46. The method of claim 43, wherein said malignant disease is selected from the group consisting of a leukemia, a lymphoma, a myeloma, a melanoma, a sarcoma, a neuroblastoma, a colon cancer, a colorectal cancer, a breast cancer, an ovarian cancer, an esophageal cancer, a synovial cell cancer and a pancreatic cancer.

47. The method of claim 34, wherein said genetically modified veto cells are non-syngeneic with the subject.

48-49. (canceled)

50. The method of claim 34, wherein said the subject is a human subject.


SEQUENCE LISTING (2 sequences)

SEQ ID NO: 1 - protein, 393 aa, Artificial Sequence
  Amino acid sequence of N29-cd28-FcR gamma human
MDFQVQIFSFLLISASVIMSRGDIVMTQSPKFMSTSVGDRISVTCKASQDVGPNVAWYQQKPGQSPKPLIYSASYLYNGVPDRFTGSGSGTDFSLTISNVQSDDLAEYFCQQYNTYPFTFGGGTKLEIKGSTSGSGKSSEGKGEVQLEESGGGLVQPKGSLKLSCAASGFTFNTYAMNWVRQAPGKGLEWIVRIRSKSNNYATYYVDSVKDRFTISRDDSQSMLYLQMNNLKTEDTAMYYCVTSYYDYDKVLFAYWGQGTTVTVKGAAAKGKHLCPSPLFPGPSKPFWVLVVVGGVLACYSLLVTVAFIIFWVRSKRSRLLHSDYMNMTPRRPGPTRKHYQPYAPPRDFAAYRSQVRKAAITSYEKSDGVYTGLSTRNQETYETLKHEKPPQL

SEQ ID NO: 2 - DNA, 1177 bp, Artificial Sequence
  Nucleic acid sequence of the N29 CAR (N29-cd28-FcR gamma) human
atggattttcaggtgcagattttcagcttcctgctaatcagtgcctcagtcataatgtctagaggagatattgtgatgacccagtctccaaaattcatgtccacatcagtaggagacaggatcagcgtcacctgcaaggccagtcaagatgtgggtcctaatgtagcctggtatcaacagaaaccagggcaatctcctaaaccactgatttactcggcatcctacctatataatggagtccctgatcgcttcacaggcagtggatctgggacagatttctctctcaccatcagcaatgtgcagtctgatgacttggcagagtatttctgtcagcaatataacacctatccgttcacgttcggagggggcaccaagctggaaatcaaagggtcgacttccggtagcggcaaatcctctgaaggcaaaggtgaggtgcagctggaggagtctggtggaggattggtgcagcctaaagggtcattgaaactctcatgtgcagcctctggattcaccttcaatacctacgccatgaactgggtccgccaggctccaggaaagggtttggaatggattgttcgcataagaagtaaaagtaataattatgcaacatattatgtcgattcagtgaaagacaggttcaccatctccagagatgattcacaaagcatgctctatctgcaaatgaacaacttgaaaactgaggacacagccatgtattactgtgtgacttcttactatgattacgacaaggtcctgtttgcttactggggccaagggaccacggtcaccgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatagatctcaagtgcgaaaggcagctataaccagctatgagaaatcagatggtgtttacacgggcctgagcaccaggaaccaggagacttacgagactctgaagcatgagaaaccaccacagtagctttagactcgag